(19) United States
(12) Patent Application Publication
(10) Pub. No.: US 2024/0424028 A1
(43) Pub. Date: Dec. 26, 2024

(54) NEURAL STEM CELLS AND USES THEREOF

(71) Applicant: AAL Scientifics, Inc., New York, NY (US)

(72) Inventor: Piero Anversa, New York, NY (US)

(21) Appl. No.: 18/737,488

(22) Filed: Jun. 7, 2024

Related U.S. Application Data
(62) Division of application No. 16/083,011, filed on Sep. 7, 2018, now abandoned.

(51) Int. Cl. A61K 35/30; A61K 9/00; A61K 38/18; A61P 25/00; C12N 5/0797

(52) U.S. Cl. CPC A61K 35/30; A61K 38/18; A61P 25/00; C12N 5/0623; A61K 9/0024; C12N 2501/115; C12N 2501/12; C12N 2501/125; C12N 2501/13

(57) ABSTRACT

Embodiments of the invention relate to stem cells and their therapeutic use in the treatment and/or prevention of neurological diseases or disorders. Provided herein are compositions comprising c-kit positive neural stem cells and methods of preparing and using c-kit positive neural stem cells for the treatment and/or prevention of neurological diseases or disorders.

### CROSS-REFERENCE TO RELATED APPLICATION

This application is a division of U.S. Nonprovisional application Ser. No. 16/083,011, filed Sep. 7, 2018. U.S. Nonprovisional application Ser. No. 16/083,011 is a National Stage Entry of PCT/US2017/020898, filed Mar. 6, 2017, which claims benefit of priority under 35 U.S.C. 119 (e) to U.S. Provisional Application No. 62/305,734, filed Mar. 9, 2016. The contents of U.S. Nonprovisional application Ser. No. 16/083,011, PCT/US217/020898, and U.S. Provisional Application No. 62/305,734 are incorporated herein by reference in their entireties.

### FIELD OF THE INVENTION

The present disclosure relates to stem cells and their therapeutic use in the treatment and/or prevention of neurological diseases or disorders. Provided herein are compositions comprising c-kit positive neural stem cells and methods of preparing and using c-kit positive neural stem cells for the treatment and/or prevention of neurological diseases or disorders.

### BACKGROUND OF THE INVENTION

The brain is a complex organ, which along with the spinal cord, is responsible for an individual's cognitive, emotional, social and motor capabilities. Neurons that specialize in different kinds of brain functions are supported by glial cells. The proper functioning of the brain is dependent upon the electrical signaling amongst neurons, and any insult to neurons or glial cells can lead to malfunctions in the brain and/or spinal cord. Neurological diseases and disorders associated with damaged neural tissue include Alzheimer's disease, Huntington's disease, amyotrophic lateral sclerosis (ALS), multiple sclerosis, Parkinson's disease, stroke and Batten disease.

Alzheimer's disease is caused by cell death in several areas of the brain. It is a progressive disorder that leads to loss of memory and cognitive abilities, and currently, no cure exists. Ultimately, Alzheimer's is fatal. The hallmarks of a brain afflicted by Alzheimer's are the beta-amyloid plaques that accumulate in the spaces between nerve cells and the tau tangles that build up inside cells. Research into therapies has focused on reducing the plaques and/or tangles.

Huntington's disease (HD) is a hereditary, degenerative brain disorder for which there is currently no cure, Huntington's disease is caused by expansion of a trinucleotide repeat in the Huntingtin gene. The gene expansion somehow leads to damage of nerve cells in areas of the brain including the basal ganglia and cerebral cortex. This leads to gradual physical, mental and emotional changes.

In amyotrophic lateral sclerosis (ALS, also known as Lou Gehrig's disease or motor neuron disease), nerve cells that control movement, located both in the spinal cord and in the brain, degenerate and die. As a result, the muscles to which those nerve cells were connected eventually weaken and waste away. Patients lose their strength and the ability to move their arms, legs and body. Eventually the muscles in the diaphragm and chest wall fail, and the patient becomes unable to breathe without support.

Multiple sclerosis is an inflammatory autoimmune-mediated disease in which the patient's immune system destroys myelin, the sheath that envelops and protects the nerves. As a result, the flow of information in the brain and spinal cord is interrupted. Ultimately, the actual nerve cells are affected and die. Patients with multiple sclerosis show a variety of symptoms involving the central nervous system, including spasms, difficulty walking, bladder and bowel problems and fatigue.

Parkinson's disease is a chronic and progressive movement disorder that occurs as a result of a gradual loss of dopaminergic neurons in an area of the brain called the substantia nigra. Patients with Parkinson's disease have difficulty in moving freely, holding a posture, talking and writing due to lack of dopamine. Individuals with Parkinson's disease have clumps of alpha synuclein protein, also called Lewy Bodies, in the mid-brain, brain stem and/or olfactory bulb.

Stroke is caused by a blockage of the blood supply to a region of the brain (ischemic stroke) or when a blood vessel in the brain bursts, spilling blood into the spaces surrounding brain cells (hemorrhagic stroke). Brain cells die when they no longer receive oxygen and nutrients from the blood or there is sudden bleeding into or around the brain. Depending on the area of the brain that is affected, several functions may be impaired, including walking, talking and cognitive ability.

Batten disease (also known as Spielmeyer-Vogt-Sjogren-Batten disease) is a fatal, inherited disorder of the nervous system that typically begins in childhood. Affected children suffer cognitive impairment, worsening seizures, and progressive loss of sight and motor skills. Batten disease is the most common form of a group of disorders called the neuronal ceroid lipofuscinoses (NCLs). Lipofuscins (lipopigments), composed of fats and proteins, build up in cells of the brain and the eye as well as in skin, muscle, and many other tissues. To date, eight genes have been linked to the varying forms of NCL.

Thus, there are a variety of neurological diseases and disorders that would benefit from therapy that would allow repair, reconstitution, regeneration or protection from further damage of cells within damaged neural tissue. However, isolation and expansion of neural stem cells from neural tissue in sufficient numbers for stem cell therapy remain a challenge. Thus, there is a need in the art to identify markers of neural stem cells that can be used to isolate such stem cells that can be expanded and used in therapy of neurological diseases or disorders.

### SUMMARY OF THE INVENTION

Embodiments of the invention relate to stem cells and methods of preparing and using them.

Embodiments of the present invention are based on the discovery of a population of c-kit positive cells in neural tissues that have characteristics typical of a stem cell. The fundamental properties of stem cells are self-renewal, clonogenicity and muitipotentiality in vitro and in vivo. The c-kit positive cells may comprise lineage-negative cells, progenitor cells and/or lineage-positive cells.

Embodiments of the present invention provide solutions to the problem of replacing damaged neural cells and/or protecting neural cells from further damage by neurological diseases or disorders such as, but not limited to, stroke, brain hemorrhage, spinal cord injury, Huntington's disease, Parkinson's disease, Alzheimer's disease, amyotrophic lateral sclerosis (ALS), multiple sclerosis (MS), Batten disease and/or ataxia telangiectasia. Specifically, the problems are solved by implanting neural stem cells to defective and/or damaged neural tissue in order to promote neural tissue repair and regeneration and to treat or prevent neurological diseases or disorders such as, but not limited to, stroke, brain hemorrhage, spinal cord injury, Huntington's disease, Parkinson's disease, Alzheimer's disease, amyotrophic lateral sclerosis (ALS), multiple sclerosis (MS), Batten disease and/or ataxia telangiectasia in a subject in need thereof.

Accordingly, in one aspect, the invention provides a method of treating or preventing a neurological disease or disorder in a subject in need thereof comprising administering isolated neural stem cells to the subject, wherein the neural stem cells are isolated from a neural tissue specimen and are c-kit positive. In one embodiment, the neural stem cells are adult neural stem cells. In another embodiment, the neural tissue specimen is obtained from the subject. In another embodiment, the neural stem cells are from the dentate gyrus of the neural tissue specimen. In a further embodiment, the neural stem cells are from the sub ventricular zone of the neural tissue specimen.

In one embodiment of a method of treating or preventing a neurological disease or disorder in a subject in need thereof, the isolated neural stem cells comprise lineage-negative cells. In another embodiment, the isolated neural stem cells comprise progenitor cells. In another embodiment, the progenitor cells express Sox2. In a further embodiment, the isolated neural stem cells comprise lineage-positive cells. In yet another embodiment, the lineage-positive cells express beta III tubulin, NeuN and/or glial fibrillary acidic protein (GFAP).

In one embodiment of a method of treating or preventing a neurological disease or disorder in a subject in need thereof, said isolated neural stem cells are expanded in culture prior to administration to the subject. In another embodiment, the isolated neural stem cells are exposed to one or more cytokines and/or growth factors prior to administration to the subject. In yet another embodiment, the isolated neural stem cells are exposed to Stem Cell Factor (SCF), insulin-like growth factor 1 (IGF-1), hepatocyte growth factor (HGF), basic fibroblast growth factor (bFGF) and/or nerve growth factor (NGF) prior to administration to the subject.

In one embodiment, the isolated neural stem cells are administered to the subject through vessels or directly to the tissue. In another embodiment, the isolated neural stem cells are administered to the subject by direct injection and/or by a catheter system,

In one embodiment of a method of treating or preventing a neurological disease or disorder in a subject in need thereof, the neurological disease or disorder is stroke. In another embodiment, the neurological disease or disorder is brain hemorrhage. In another embodiment, the neurological disease or disorder is a neurodegenerative disease. In yet another embodiment, the neurodegenerative disease is Huntington's disease, Parkinson's disease, Alzheimer's disease, amyotrophic lateral sclerosis (ALS), multiple sclerosis (MS), Batten disease and/or ataxia telangiectasia.

In another aspect, the invention provides a pharmaceutical composition comprising a therapeutically effective amount of isolated neural stem cells and a pharmaceutically acceptable carrier for repairing and/or regenerating damaged neural tissue, wherein said isolated neural stem cells are c-kit positive. In some embodiments, the neural stem cells are adult neural stem cells. In another embodiment, the isolated neural stem cells are clonogenic, multipotent and self-renewing.

In one embodiment of a pharmaceutical composition, the neural stem cells are isolated from the dentate gyrus of neural tissue. In another embodiment, the neural stem cells are isolated from the sub ventricular zone of neural tissue. In another embodiment, the isolated neural stem cells are human cells. In a further embodiment, the isolated neural stem cells are autologous.

In one embodiment of a pharmaceutical composition, the isolated neural stem cells comprise lineage-negative cells. In another embodiment, the isolated neural stem cells comprise progenitor cells. In another embodiment, the progenitor cells express Sox2. In a further embodiment, the isolated neural stem cells comprise lineage-positive cells. In yet another embodiment, the lineage-positive cells express beta III tubulin, NeuN and/or GFAP.

In one embodiment of a pharmaceutical composition, the composition comprises about 106 isolated neural stem cells. In another embodiment, the isolated neural stem cells are cultured and expanded in vitro. In another embodiment, the composition further comprises one or more cytokines and/or growth factors. In a further embodiment, the composition further comprises SCF, IGF-1, HGF, bFGF and/or NGF.

In one embodiment, the composition is formulated for catheter-mediated or direct injection.

In one embodiment of a pharmaceutical composition, the isolated neural stem cells are capable of forming neurospheres, wherein each neurosphere comprises a core and one or more outer layers. In another embodiment, the neurospheres comprise lineage-negative cells. In another embodiment, the lineage-negative cells are in the core of each neurosphere. In a further embodiment, the neurospheres comprise progenitor cells. In some embodiments, the progenitor cells express Sox2. In another embodiment, the neurospheres comprise lineage-positive cells. In a further embodiment, the lineage-positive cells are in one or more outer layers of each neurosphere. In yet another embodiment, the lineage-positive cells express beta III tubulin, NeuN and/or GFAP.

In another aspect, the invention provides a method of isolating resident neural stem cells from neural tissue comprising: (a) culturing a tissue specimen from said neural tissue in culture, thereby forming a tissue explant; (b) selecting cells from the cultured explant that are c-kit positive, and (c) isolating said c-kit positive cells, wherein said isolated c-kit positive cells are resident neural stem cells.

In one embodiment, said isolated c-kit positive cells are from the dentate gyrus of the neural tissue. In another embodiment said isolated c-kit positive cells are from the subventricular zone of the neural tissue.

In one embodiment of a method of isolating resident neural stem cells from neural tissue, the isolated c-kit positive cells comprise lineage-negative cells. In another embodiment, the isolated neural c-kit positive cells comprise progenitor cells. In another embodiment, the progenitor cells express Sox2. In a further embodiment, the isolated c-kit positive cells comprise lineage-positive cells. In yet another embodiment, the lineage-positive cells express beta III tubulin, NeuN and/or GFAP.

In one embodiment, a method of isolating resident neural stem cells from neural tissue further comprises expanding said isolated c-kit positive cells in culture. In another embodiment, the method further comprises exposing said isolated c-kit positive cells to one or more cytokines and/or growth factors in culture. In yet another embodiment, the method further comprises exposing said isolated c-kit positive cells to SCF, IGF-1, HGF, bFGF and/or NGF in culture.

In another aspect, the invention provides a method of repairing and/or regenerating damaged neural tissue in a subject in need thereof comprising: extracting neural stem cells from healthy neural tissue; culturing and expanding said neural stem cells, said neural stem cells being c-kit positive stem cells; and administering a dose of said extracted and expanded neural stem cells to an area of damaged neural tissue in the subject effective to repair and/or regenerate the damaged neural tissue.

In one embodiment of a method of repairing and/or regenerating damaged neural tissue in a subject in need thereof, the extracted and expanded c-kit positive stem cells are from the dentate gyrus of the healthy neural tissue. In another embodiment, the extracted and expanded c-kit positive stem cells are from the subventricular zone of the healthy neural tissue.

In one embodiment of a method of repairing and/or regenerating damaged neural tissue in a subject in need thereof, the extracted and expanded c-kit positive stem cells comprise lineage-negative cells. In another embodiment, the extracted and expanded c-kit positive stem cells comprise progenitor cells. In a further embodiment, the extracted and expanded c-kit positive stem cells comprise lineage-positive cells. In yet another embodiment, the extracted and expanded c-kit positive stem cells express beta Ill tubulin, NeuN and/or GFAP.

In one embodiment of a method of repairing and/or regenerating damaged neural tissue in a subject in need thereof, the extracted and expanded c-kit positive stem cells are exposed to one or more cytokines and/or growth factors in culture prior to administration to the damaged neural tissue. In yet another embodiment, the extracted and expanded c-kit positive stem cells are exposed to SCF, IGF-1, HGF, bFGF and/or NGF prior to administration to the damaged neural tissue.

In one embodiment of a method of repairing and/or regenerating damaged neural tissue in a subject in need thereof, the extracted and expanded c-kit positive stem cells are administered by catheter-mediated or direct injection.

In one embodiment of all aspects of the compositions and methods described, the neural tissue is from a human. In another embodiment of all aspects of the compositions and methods described, the neural tissue is an adult neural tissue. In another embodiment of all aspects of the compositions and methods described, the isolated neural stem cells are clonogenic, multipotent and self-renewing. In another embodiment of all aspects of the compositions and methods described, the c-kit-positive cells are clonogenic, multipotent and self-renewing. In another embodiment of all aspects of the compositions and methods described, the isolated neural stem cells comprise lineage-negative cells. In another embodiment of all aspects of the compositions and methods described, the isolated neural stem cells comprise progenitor cells. In a further embodiment of all aspects of the compositions and methods described, the isolated neural stem cells comprise lineage-positive cells. In yet another embodiment of all aspects of the compositions and methods described, the lineage-positive cells express beta III tubulin, NeuN and/or GFAP. In another embodiment of all aspects of the compositions and methods described, the neural stem cells are autologous. In another embodiment of all aspects of the compositions and methods described, the neural stem cells are allogeneic.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows representative immunolabeling in situ of dentate gyrus of mouse brain, c-kit is labeled green and GFAP is labeled red. Cell nuclei are stained with DAPI.

FIG. 1B shows representative immunolabeling in situ of a mouse brain tissue section. c-kit is labeled red and GFAP is labeled green. Cell nuclei are stained with DAPI.

FIG. 2 shows representative immunolabeling in situ of a mouse brain tissue section, c-kit is labeled green and Sox2 is labeled white. Cell nuclei are stained with DAPI.

FIG. 3A shows representative immunolabeling in situ of a mouse brain tissue section. c-kit is labeled green and beta III tubulin is labeled red. Cell nuclei are stained with DAPI. The red arrow points to a c-kit positive cell that expresses both beta III tubulin and NeuN.

FIG. 3B shows representative immunolabeling in situ of a mouse brain tissue section, c-kit is labeled green and NeuN is labeled white. Cell nuclei are stained with DAPI.

FIGS. 4A and 4B show representative images of neurospheres derived from unsorted cells after 7-14 days in culture.

FIG. 5 shows compact and well-separated neurospheres following stimulation with the ligand of the c-kit receptor, SCF.

FIG. 6A-6B shows immunolabeling of neurospheres at passage 2. Cell nuclei are stained with DAPI. One neurosphere expresses c-kit (green), NeuN (gray) and GFAP (red). The merge of signals for the three markers are shown in FIG. 6B, while FIG. 6A shows the individual marker signals. The other neurosphere is negative for all three markers and the nuclei can be seen by DAPI staining.

FIG. 7 shows immunolabeling of a neurosphere at passage 4. Cell nuclei are stained with DAPI. The core of the neurosphere contains c-kit positive (green), lineage-negative cells, while the outer layer of the neurosphere expresses the neuronal marker GFAP (red).

FIG. 8 shows passage 4 neurospheres transferred to adherent dishes.

FIGS. 9A and 9B show immunolabeling of passage 4 neurospheres that have been transferred to adherent dishes, c-kit positive (green) cells partly co-express lineage markers of neural cells (GFAP, red, NeuN, gray).

### DETAILED DESCRIPTION OF THE INVENTION

Embodiments of the present invention are based on the discovery of a population of c-kit positive cells in neural tissues that have characteristics typical of a stem cell. The fundamental properties of stem cells are the ability to self-renew, i.e., make more of stem cells, clonogenicity and multipotentiality in vitro and in vivo. Prior to this discovery, there has been no recognition or isolation of one cell type from neural tissues that exhibits all three characteristics of a stem cell.

As it is well known, stem cells, by virtue of their properties, give rise to all the cells and tissues of the body. Therefore, stem cells can be used to repair or speed up the repair of damaged and/or defective neural tissue. If a sufficient amount of neural stem cells (NSCs) can be obtained, this amount of NSCs can be used to repair damaged and/or defective neural tissue by building new tissues in the brain and/or spinal cord. In defective and/or damaged neural tissue, there may be few or absent NSCs. Since NSCs self-renew, the implanted NSCs will colonize and populate niches in the defective and/or damaged neural tissue. By being clonal and multipotent, the implanted NSCs will also divide and differentiate to produce all new neural cells and tissues. Therefore, a population of isolated NSCs or a composition comprising a population of isolated NSCs can be used for treatment or prevention of a neurological disease or disorder in a subject.

Accordingly, in one embodiment, the invention provides a population of isolated cells from a sample of neural tissue, wherein the population of isolated cells contains c-kit positive NSCs. This population of c-kit-positive NSCs can be enriched and expanded significantly.

In one embodiment, provided herein is a pharmaceutical composition comprising a therapeutically effective amount of isolated and expanded neural stem cells and a pharmaceutically acceptable carrier for repairing and/or regenerating damaged neural tissue, wherein said isolated neural stem cells are c-kit positive. In some embodiments, the neural stem cells are adult neural stem cells. In another embodiment, the isolated neural stem cells are clonogenic, multipotent and self-renewing. In some embodiments, the neural stem cells are isolated from the dentate gyrus of neural tissue. In another embodiment, the neural stem cells are isolated from the sub ventricular zone of neural tissue. In another embodiment, the isolated neural stem cells are human cells. In a further embodiment, the isolated neural stem cells are autologous.

In one embodiment, the isolated neural stem cells comprise lineage-negative cells. In another embodiment, the isolated neural stem cells comprise progenitor cells. In another embodiment, the progenitor cells express Sox2. In a further embodiment, the isolated neural stem cells comprise lineage-positive cells. In yet another embodiment, the lineage-positive cells express beta III tubulin, NeuN and/or GFAP.

In one embodiment, the composition comprises about 106 isolated neural stem cells. In another embodiment, the isolated neural stem cells are cultured and expanded in vitro. In another embodiment, the composition further comprises one or more cytokines and/or growth factors. In a further embodiment, the composition further comprises SCF, IGF-1, HGF, bFGF and/or NGF.

In one embodiment, the composition is formulated for catheter-mediated or direct injection.

In one embodiment, the isolated neural stem cells are capable of forming neurospheres, wherein each neurosphere comprises a core and one or more outer layers. In another embodiment, the neurospheres comprise lineage-negative cells. In another embodiment, the lineage-negative cells are in the core of each neurosphere. In a further embodiment, the neurospheres comprise progenitor cells. In some embodiments, the progenitor cells express Sox2. In another embodiment, the neurospheres comprise lineage-positive cells. In a further embodiment, the lineage-positive cells are in one or more outer layers of each neurosphere. In yet another embodiment, the lineage-positive cells express beta III tubulin, NeuN and/or GFAP.

In one embodiment, provided herein is a composition for use in the manufacture of a medicament for the treatment and/or prevention of a neurological disease or disorder in a subject, the composition comprising an enriched population of isolated c-kit positive NSCs from a neural tissue sample. In another embodiment of this composition, the composition further comprises a pharmaceutically acceptable carrier.

In one embodiment, the invention provides a method of isolating resident neural stem cells from neural tissue comprising: (a) culturing a tissue specimen from said neural tissue in culture, thereby forming a tissue explant; (b) selecting cells from the cultured explant that are c-kit positive, and (c) isolating said c-kit positive cells, wherein said isolated c-kit positive cells are resident neural stem cells.

In one embodiment, said isolated c-kit positive cells are from the dentate gyrus of the neural tissue. In another embodiment, said isolated c-kit positive cells are from the sub ventricular zone of the neural tissue.

In one embodiment, the isolated c-kit positive cells comprise lineage-negative cells. In another embodiment, the isolated neural c-kit positive cells comprise progenitor cells. In another embodiment, the progenitor cells express Sox2. In a further embodiment, the isolated c-kit positive cells comprise lineage-positive cells. In yet another embodiment, the lineage-positive cells express beta III tubulin, NeuN and/or GFAP.

In one embodiment a method of isolating resident neural stem cells from neural tissue further comprises expanding said isolated c-kit positive cells in culture. In another embodiment, the method further comprises exposing said isolated c-kit positive cells to one or more cytokines and/or growth factors in culture. In yet another embodiment, the method further comprises exposing said isolated c-kit positive cells to SCF, IGF-1, HGF, bFGF and/or NGF in culture.

In one embodiment, the invention provides a method of obtaining a population of isolated cells substantially enriched for c-kit positive NSCs, the method comprising cryopreserving a specimen of neural tissue obtained from a subject; thawing the cryopreserved specimen at a later date; selecting one or more c-kit positive cells from the specimen of neural tissue; and proliferating the selected c-kit positive cells in a culture medium.

In one embodiment, the invention provides a method of proliferating a population of isolated cells substantially enriched for c-kit positive NSCs, the method comprising selecting one or more c-kit positive cells from a neural tissue sample; introducing the one or more c-kit positive selected cells to a culture medium; and proliferating the selected c-kit positive cells in the culture medium.

In another embodiment, the invention provides methods of use of this population of isolated cells that is substantially enriched for c-kit positive NSCs or use of a pharmaceutical composition comprising an enriched population of isolated c-kit positive NSCs, for example, in the repair, regeneration and/or treatment of neurological diseases or disorders such as stroke, brain hemorrhage, spinal cord injury, Huntington's disease, Parkinson's disease, Alzheimer's disease, amyotrophic lateral sclerosis (ALS), multiple sclerosis (MS), Batten disease and/or ataxia telangiectasia. Without wishing to be bound by theory, the inventors consider that the c-kit-positive-cells identified in neural tissue may represent the source of the specialized cells in the brain, such as cholinergic neurons, GABAergic (gamma aminobutyric acid) neurons, glutamatergic neurons, dopaminergic neurons, serotonergic neurons, motor neurons, interneurons, astrocytes, oligodendrocytes and/or microglia. Hence, in one embodiment, a population of isolated c-kit positive NSCs which have been expanded in vitro can be transplanted or implanted into affected/damaged neural tissue. The c-kit positive NSCs then take up residence in the neural tissue, grow and differentiate into the various types of tissues normally found in brain or spinal cord, and restore and/or reconstitute the specialized cells of the brain and/or spinal cord. The goal is to replace some of the damaged neural tissue due to disease in the affected tissue. The replacement neural tissue serves to supplement existing or remaining neural tissue in the affected subject so that over all there is enough tissue for adequate functions of the brain and/or spinal cord to ameliorate, treat and/or prevent neurological disease or disorder in that subject.

In one embodiment, differentiated c-kit-positive NSCs can be transplanted into an animal model of a particular neurological disease or disorder to establish whether NSCs can differentiate into healthy neural cells to thus ameliorate, treat and/or prevent neurological disease or disorder in the animal. For example, c-kit-positive NSCs can be transplanted into an animal models of e.g., Parkinson's Disease, ALS, and stroke as described in Adami at el. Front Cell Dev Biol. 2014; 2:17.

Adult stem cell transplantation has emerged as a new alternative to stimulate repair of injured tissues and organs. In the past decade, some studies in animals and humans have documented the ability of adult bone marrow-derived stem cells, i.e., hematopoietic stem cells, to differentiate into an expanding repertoire of non-hematopoietic cell types, including brain, skeletal muscle, chondrocytes, liver, endothelium, and heart.

There is no literature that demonstrates the presence of bona fide multipotent tissue-specific adult c-kit positive neural stem cells in brain and the use of these NSCs to treat or prevent neurological diseases or disorders in patients. The advantage of the present invention is that the NSCs used in treatment or prevention of neural diseases or disorders can be autologous cells which will greatly increase success rate of treatment or prevention. A portion of a patient's neural tissue is removed surgically, e.g., during a biopsy. As little as one cubic centimeter is sufficient. The piece of tissue is treated to release single cells from the connective tissue. Using the stem cell marker, c-kit, as an indication of stem cells, c-kit positive cells are selected. The c-kit positive NSCs are then expanded in vitro to obtain sufficient number of cells required for treatment or prevention. When there are enough cells, the cells are harvested and injected back into the same patient or a genetically matched patient with respect to the donor of the NSCs. At each transitional step, e.g., between selection and expansion or between expansion and implanting, the NSCs can be optionally cryopreserved. In one embodiment, the patient gets back the patient's own NSCs that have been selected and expanded in vitro. In another embodiment, the patient gets the NSCs derived from a genetically matched donor. In some embodiments, this method can also be extended to any mammal that has neural tissue, e.g., cat, dog, horse, monkey etc.

Accordingly, the invention provides a method of treating or preventing a neurological disease or disorder in a subject in need thereof comprising administering isolated neural stem cells to the subject, wherein the neural stem cells are isolated from a neural tissue specimen and are c-kit positive. In one embodiment, the neural stem cells are adult neural stem cells. In another embodiment, the neural tissue specimen is obtained from the subject. In another embodiment, the neural stem cells are from the dentate gyrus of the neural tissue specimen. In a further embodiment, the neural stem cells are from the sub ventricular zone of the neural tissue specimen,

In one embodiment, the isolated neural stem cells comprise lineage-negative cells. In another embodiment, the isolated neural stem cells comprise progenitor cells. In another embodiment, the progenitor cells express Sox2. In a further embodiment, the isolated neural stem cells comprise lineage-positive cells. In yet another embodiment, the lineage-positive cells express beta Ill tubulin, NeuN and/or GFAP.

In one embodiment, said isolated neural stem cells are expanded in culture prior to administration to the subject. In another embodiment, the isolated neural stem cells are exposed to one or more cytokines and/or growth factors prior to administration to the subject. In yet another embodiment, the isolated neural stem cells are exposed to SCF, IGF-1, HGF, bFGF and/or NGF prior to administration to the subject.

In one embodiment, the isolated neural stem cells are administered to the subject through vessels or directly to the tissue. In another embodiment, the isolated neural stem cells are administered to the subject by injection and/or by a catheter system.

In one embodiment, the neurological disease or disorder is stroke. In another embodiment, the neurological disease or disorder is brain hemorrhage. In another embodiment, the neurological disease or disorder is a neurodegenerative disease. In yet another embodiment, the neurodegenerative disease is Huntington's disease, Parkinson's disease, Alzheimer's disease, amyotrophic lateral sclerosis (ALS), multiple sclerosis (MS), Batten disease and/or ataxia telangiectasia.

In one embodiment, provided here is a method for treating and/or preventing a neurological disease or disorder in a subject in need thereof, the method comprising administering a composition comprising a population of c-kit positive NSCs described herein to the subject.

In another embodiment, the invention provides a method for treating and/or preventing a neurological disease or disorder in a subject in need thereof, comprising obtaining a sample of neural tissue from a subject; extracting a population of c-kit positive NSCs from the neural tissue sample; expanding the selected c-kit positive NSCs in vitro to increase the numbers of such NSCs; and administering the expanded population of c-kit positive NSCs to the subject to repair, reconstitute or regenerate neural cells and tissues in the brain and/or spinal cord of the subject.

In another embodiment, the invention provides a method for treating or preventing a neurological disease or disorder in a subject in need thereof, the method comprising obtaining neural tissue from a first subject; extracting a population of c-kit positive NSCs from the neural tissue sample, expanding the population of c-kit positive NSCs; and administering the population of c-kit positive NSCs to a second subject for the c-kit NSCs to take up residence in the brain and/or spinal cord and repair, reconstitute, and/or regenerate neural cells and tissues in the brain and/or spinal cord of the second subject.

In another embodiment, the invention provides a method of repairing and/or regenerating damaged neural tissue in a subject in need thereof comprising: extracting neural stem cells from healthy neural tissue; culturing and expanding said neural stem cells, said neural stem cells being c-kit positive stem cells; and administering a dose of said extracted and expanded neural stem cells to an area of damaged neural tissue in the subject effective to repair and/or regenerate the damaged neural tissue.

In one embodiment of a method of repairing and/or regenerating damaged neural tissue in a subject in need thereof, the extracted and expanded c-kit positive stem cells are from the dentate gyrus of the healthy neural tissue. In another embodiment, the extracted and expanded c-kit positive stem cells are from the sub ventricular zone of the healthy neural tissue.

In one embodiment of a method of repairing and/or regenerating damaged neural tissue in a subject in need thereof, the extracted and expanded c-kit positive stem cells comprise lineage-negative cells. In another embodiment, the extracted and expanded c-kit positive stem cells comprise progenitor cells. In a further embodiment, the extracted and expanded c-kit positive stem cells comprise lineage-positive cells. In yet another embodiment, the extracted and expanded c-kit positive stem cells express beta III tubulin, NeuN and/or GFAP.

In one embodiment of a method of repairing and/or regenerating damaged neural tissue in a subject in need thereof, the extracted and expanded c-kit positive stem cells are capable of generating one or more neural cell types. In another embodiment, the one or more neural cell types comprise cholinergic neurons. In another embodiment, the one or more neural cell types comprise GABAergic neurons. In another embodiment, the one or more neural cell types comprise glutamatergic neurons. In another embodiment, the one or more neural cell types comprise dopaminergic neurons. In a further embodiment, the one or more neural cell types comprise serotonergic neurons. In another embodiment, the one or more neural cell types comprise motor neurons. In a further embodiment, the one or more neural cell types comprise interneurons. In another embodiment, the one or more neural cell types comprise astrocytes. In yet another embodiment, the one or more neural cell types comprise oligodendrocytes. In some embodiments, the one or more neural cell types comprise microglia.

In one embodiment of a method of repairing and/or regenerating damaged neural tissue in a subject in need thereof, the extracted and expanded c-kit positive stem cells are exposed to one or more cytokines and/or growth factors in culture prior to administration to the damaged neural tissue. In yet another embodiment, the extracted and expanded c-kit positive stem cells are exposed to SCF, IGF-1, HGF, bFGF and/or NGF prior to administration to the damaged tissue.

In one embodiment of a method of repairing and/or regenerating damaged neural tissue in a subject in need thereof, the extracted and expanded c-kit positive stem cells are administered by catheter-mediated or direct injection.

In one embodiment of all aspects of the compositions and methods described, the c-kit positive NSCs that make up predominantly the population of isolated cells have self-renewal capability, clonogenicity and multipotentiality. This means that each isolated c-kit positive cell can divide to give rise to more c-kit positive cells, forming a colony in culture. When stimulated under certain conditions, each c-kit positive cell can become committed (i.e., selecting a specific cell lineage to differentiate into) and further differentiate to cells of a specific lineage, e.g., GABA neurons, dopamine neurons, motor neurons, astrocytes, oligodendrocytes (myelin-producing) and/or microglia. These cells and their progeny, upon specification and differentiation, will express the particular cell markers characteristic of the determined lineage. In addition, the committed cell and its progeny will lose the expression of c-kit,

In one embodiment of all aspects of the compositions and methods described, the neural tissue is from a human. In another embodiment of all aspects of the compositions and methods described, the human is an adult.

In one embodiment of all aspects of the described methods, the neural tissue is cryopreserved prior to selecting c-kit positive cells.

In one embodiment of all aspects of the described methods, the selection of the c-kit-positive NSCs is performed using an antibody against c-kit.

In one embodiment of all aspects of the described methods, the antibody against c-kit is a monoclonal antibody.

In one embodiment of all aspects of the described methods, the monoclonal antibody against c-kit is a mouse monoclonal IgG against an antigenic epitope of human c-kit.

In one embodiment of the any of the described methods, the antibody against c-kit is fluorochrome conjugated.

In one embodiment of all aspects of the described methods, the anti body against c-kit is conjugated to magnetic particles.

In one embodiment of all aspects of the described methods, the selection of c-kit positive cells is by flow cytometry.

In one embodiment of all aspects of the described methods, the selection is by fluorescence activated cell sorting or high gradient magnetic selection.

In another embodiment of all aspects of the compositions and methods described, the isolated neural stem cells comprise lineage-negative cells, progenitor cells and/or lineage-positive cells.

In one embodiment of all aspects of the described methods, the c-kit positive NSCs are further expanded ex vivo. In one embodiment of all aspects of the described methods, the c-kit positive NSCs are further expanded in vitro. The goal is to have a sufficiently large amount of c-kit positive NSCs for implanting to ensure successful engrafting of the implanted NSCs into niches of the damaged neural tissue. Basically, there must be sufficient cells to grow and multiply in the damaged neural tissue to provide ail the cells needed to repair and/or replace the damaged parts of the neural tissue,

In one embodiment of all aspects of the described methods, the c-kit positive NSCs are at least double in number after the expansion or proliferation step. In some embodiments of all aspects of the described methods, it is desirable that the number of c-kit positive cells, upon expansion or proliferation, is increased by at least 5 fold, 10 fold, 20 fold, 50 fold, 100 fold, 200 fold, 500 fold, 1000 fold, 2000 fold, 5000 fold, 10,000 fold, 20,000 fold, 50,000 fold or more at the end of the proliferation phase. The number of cells in a culture can be determined by any methods known in the art, e.g., by using a coulter counter. These methods are well known to those skilled in the art.

In one embodiment of all aspects of the described methods, the selected c-kit positive NSCs are cryopreserved for storage prior to expansion.

In another embodiment of all aspects of the described methods, the expanded NSCs are cryopreserved for storage purposes. When needed, the frozen cells are thawed and then used for implanting into a subject in need thereof.

In one embodiment of all aspects of the described methods, the method further comprises cryopreserving the population of isolated c-kit positive NSCs.

For a person who has been newly diagnosed with a neurological disease or disorder, if a biopsy sample of the subject's neural tissue was obtained for the diagnosis, a population of c-kit positive NSCs can be prepared according to the methods described here and the NSCs can then be cryopreserved for future use in the event that the disease had progressed to an advanced stage such that the person needed neural stem cell therapy.

Similarly, a person who is at risk of developing a neurological disease or disorder can benefit from early preparation of a population of c-kit NSCs from the person's own neural tissue and cryopreserving the NSCs. For example, a person with a genetic disposition to Huntington's disease would benefit. Huntington's Disease (HD) is a hereditary, degenerative brain disorder for which there is currently no cure. Huntington's disease is caused by expansion of the Huntingtin gene due to repeats of a CAG trinucleotide sequence. On average the larger the gene expansion, the earlier the age of onset of the disease. Other people at risk of developing neurological diseases or disorders include, but are not limited to: individuals with family members having early onset familial Alzheimer's disease, individuals having the ApoE susceptibility gene for Alzheimer's disease, and individuals with family members having amyotrophic lateral sclerosis (ALS) or individuals carrying genes known to cause ALS.

In some embodiments of all aspects of the therapeutic methods, treating and treatment includes “restoring structural and functional integrity” to a damaged neural tissue in a subject in need thereof.

In other embodiments of all aspects of the described methods, treating includes repairing damaged or inadequate human neural tissue. In another embodiment, treating and treatment includes repair, reconstitution, regeneration or protection from further damage, of neural cells in the damaged neural tissue.

The restoring or repairing need not be to 100% to that of the neural tissue of a healthy person. As long as there is an improvement in the symptoms in the subject, restoring or repairing has been achieved. A skilled physician would be able to assess the severity of the symptoms before and after the treatment and based on a comparison determine whether there is an improvement. Often, the subject will be able to say whether there is an improvement in the symptoms. Examples of some symptoms include, but are not limited to: memory loss, cognitive decline, motor decline, fatigue, or bladder and bowel problems.

In one embodiment of all aspects of the therapeutic methods, preventing and prevention includes slowing down the reduced functioning capacity and integrity of the neural tissue due to disease, e.g., from stroke, brain hemorrhage, spinal cord injury or a neurodegenerative disease.

In one embodiment of all aspects of the therapeutic methods, the population of c-kit positive NSCs repairs, reconstitutes, generates or protects from further damage, neural cells in the neural tissue.

In one embodiment of all aspects of the therapeutic methods, the method of treating and/or preventing a neurological disease or disorder further comprises selecting a subject who is suffering from a neurological disease or disorder prior to administering the population of cells that is substantially enriched for c-kit positive NSCs, e.g., a subject suffering from stroke, brain hemorrhage, spinal cord injury or a neurodegenerative disease.

In one embodiment of all aspects of the therapeutic methods, the method of treating and/or preventing a neurological disease or disorder further comprises selecting a subject in need of restoring the structural and functional integrity of a damaged neural tissue prior to administering the cells, e.g. a subject suffering from stroke, brain hemorrhage, spinal cord injury or a neurodegenerative disease.

In one embodiment of all aspects of the therapeutic methods, the method of treating and/or preventing a neurological disease or disorder further comprises selecting a subject in need of repair, reconstitution, regeneration or protection from further damage, of neural cells in the neural tissue, e.g., a subject suffering from stroke, brain hemorrhage, spinal cord injury or a neurodegenerative disease.

For example, the selected subjects are those who have not responded at all or well to the traditional treatment and/or one who has exhausted all therapeutic options currently known in the art for a particular form or type of a neurological disease or disorder.

In one embodiment of all aspects of the therapeutic methods for treating or preventing a neurological disease or disorder, the administration is by direct injection, by a catheter system, or a combination thereof.

In one embodiment of all aspects of the therapeutic methods for treating or preventing a neurological disease or disorder, the administration to the subject is through vessels, directly to the tissue, or a combination thereof.

In one embodiment of all aspects of the therapeutic methods for treating or preventing a neurological disease or disorder, the c-kit positive NSCs are autologous cells.

In one embodiment of all aspects of the therapeutic methods for treating or preventing a neurological disease or disorder, the c-kit positive NSCs are allogeneic cells obtained from one or more donors.

In one embodiment of all aspects of the therapeutic methods, the method further comprises administration with at least one therapeutic agent with the c-kit positive NSCs, e.g., those for treating stroke, brain hemorrhage, spinal cord injury or a neurodegenerative disease.

In one embodiment of all aspects of the therapeutic methods, the at least one therapeutic agent enhances homing, engraftment, or survival of the population of NSCs.

In one embodiment of all aspects of the therapeutic methods, the subject is a mammal, preferably a human. In another embodiment, the subject is an adult human. In one embodiment, the population of c-kit positive NSCs is a population of c-kit positive human NSCs.

### Neural Development

The nervous system arises from the ectoderm, the outermost tissue layer of the embryo. In the third week of development the neuroectoderm appears and forms the neural plate along the dorsal side of the embryo. This neural plate is the source of the majority of neurons and glial cells in the mature human. Neurons and glial cells are the main cellular components of the brain.

Three types of glial cells are found in the central nervous system (CNS): astrocytes, oligodendrocytes and microglial cells.

Astrocytes are a heterogeneous cell population which interact with neurons and blood vessels. These cells detect neuronal activity and modulate neuronal networks. An archetypal morphological feature of astrocytes is their expression of intermediate filaments, which form the cytoskeleton. The main types of astroglial intermediate filament proteins are glial fibrillary acidic protein (GFAP) and vimentin; expression of GFAP is commonly used as a specific marker for the identification of astrocytes.

Oligodendrocytes in the central nervous system produce myelin. Myelin acts as an insulator of axonal segments and is a prerequisite for the high velocity of nerve conduction. All white matter tracts contain oligodendrocytes to form myelin. There are also oligodendrocytes that are not directly connected to the myelin sheath. These satellite oligodendrocytes are preferentially found in gray matter and may serve to regulate ionic homeostasis similarly to astrocytes.

Microglial cells are the immune cells of the central nervous system and are responsible for CNS protection against various types of pathogenic factors. They can migrate to the site of damage, proliferate and become phagocytes, and they interact with the peripheral immune system by antigen presentation.

Neurons are the core components of the brain and spinal cord of the CNS, and of the ganglia of the peripheral nervous system (PNS). Neurons can connect to each other to form neural networks. Specialized types of neurons include: sensory neurons which respond to touch, sound, light and all other stimuli affecting the cells of the sensory organs that then send signals to the spinal cord and brain, motor neurons that receive signals from the brain and spinal cord to cause muscle contractions and affect glandular outputs, and interneurons which connect neurons to other neurons within the same region of the brain, or spinal cord in neural networks.

A typical neuron consists of a cell body (soma), dendrites, and an axon. Dendrites are thin structures that arise from the cell body, often extending for hundreds of micrometers and branching multiple times, giving rise to a complex “dendritic tree”. An axon (also called a nerve fiber when myelinated) is a special cellular extension that arises from the cell body and travels for a distance, as far as 1 meter in humans or even more in other species.

Neurons are electrically excitable cells that process and transmit information through electrical and chemical signals. These signals between neurons occur via specialized connections called synapses. At the majority of synapses, signals are sent from the axon of one neuron to a dendrite of another.

### Neural Stem Cells (NSCs)

Stem cells are cells that retain the ability to renew their own kind through mitotic cell division and their daughter cells can differentiate into a diverse range of specialized cell types. The two broad types of mammalian stem cells are: embryonic stem (ES) cells that are found in blastocysts, and adult stem cells that are found in adult tissues. In a developing embryo, ESs can differentiate into all of the specialized embryonic tissues. In adult organisms, adult stem cells and progenitor cells act as a repair system for the body, replenishing specialized cells, but also maintaining the normal turnover of regenerative organs, such as blood, skin or intestinal tissues. Pluripotent stem cells can differentiate into cells derived from any of the three germ layers.

In some embodiment, the term “stem cell” as used herein, refers to an undifferentiated cell which is capable of proliferation and giving rise to more progenitor cells having the ability to generate a large number of mother cells that can in turn give rise to differentiated, or differentiable daughter cells known as precursor cells. The daughter cells themselves can be induced to proliferate and produce progeny that subsequently difterentiate into one or more mature cell types, while also retaining one or more cells with parental developmental potential. In some embodiment, the term “stem cell” also refers to a subset of progenitors that have the capacity or potential, under particular circumstances, to differentiate to a more specialized or differentiated phenotype, and also retains the capacity, under certain circumstances, to proliferate without substantially differentiating,

The NSCs described herein are somatic stem cells as opposed to ESs. In a preferred embodiment, the NSCs described are adult stem cells.

In one embodiment, as used herein, the term “c-kit positive neural stem cell” or “c-kit positive NSC” encompass stem cells, progenitor cells and precursor cells, all of which are c-kit positive.

In one embodiment, as used herein, the term “c-kit positive neural stem cell” or “c-kit positive NSC” encompasses c-kit positive cells that comprise lineage-negative cells, progenitor cells and/or lineage-positive cells. In yet another embodiment, the lineage-positive cells express beta III tubulin, NeuN and/or GFAP.

Cellular differentiation is a complex process typically occurring through many cell divisions. A differentiated cell may derive from a multipotent cell which itself is derived from a multipotent cell, and so on. While each of these multipotent cells may be considered stem cells, the range of cell types each multipotent cell can give rise to may vary considerably. Some differentiated cells also have the capacity to give rise to cells of greater developmental potential. Such capacity may be natural or may be induced artificially upon treatment with various factors. In many biological instances, stem cells are “multipotent” because they can produce progeny of more than one distinct cell type, and it is required as used in this document. Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stem cells, thus maintaining some stem cells in the population as a whole, while other cells in the population give rise to differentiated progeny only.

In one embodiment, the population of isolated cells that is substantially enriched for c-kit positive cells comprises predominantly NSCs. Therefore, in one embodiment, the population of isolated cells that is substantially enriched for c-kit positive cells is referred to as a population of isolated c-kit positive NSCs. It is meant that the population of c-kit positive NSCs can include some c-kit positive lineage-negative cells, c-kit positive progenitor cells and/or c-kit positive precursor cells.

As used herein, in some embodiments, the term “a population of isolated and substantially enriched for c-kit positive NSCs” or “a population of isolated e-kit positive NSCs” encompasses a heterogeneous or homogeneous population of NSCs and/or neural progenitor cells and/or neural precursor cells. NSCs are multipotent and produce cell types of many lineages. In contrast, neural progenitor cells and neural precursor cells are lineage determinate cells. For example, if a neural progenitor cell is determinate for a glial cell lineage, i.e., will produce glial cells in the future, this neural progenitor cell will not switch and produce neuronal cells. In some embodiments, neural progenitor cells and neural precursor cells are determinate for cholinergic neurons, GABAergic neurons, glutamatergic neurons, dopaminergic neurons, serotonergic neurons, motor neurons, interneurons, astrocytes, oligodendrocytes or microglia.

A population of isolated c-kit positive NSCs comprising at least two different cell types is referred to herein as a “heterogeneous population”. It is also contemplated herein that neural stem cells or neural progenitor cells are isolated and expanded ex vivo prior to transplantation. A population of isolated c-kit positive NSCs comprising only one cell type (e.g., neuronal cells) is referred to herein as a “homogeneous population of cells”.

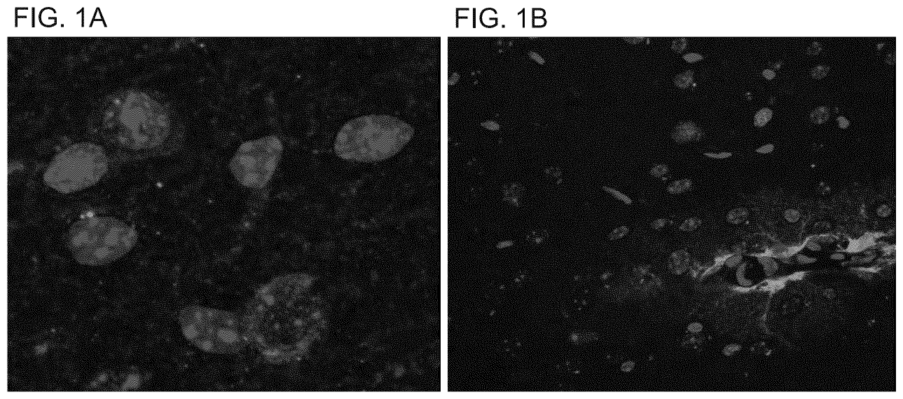

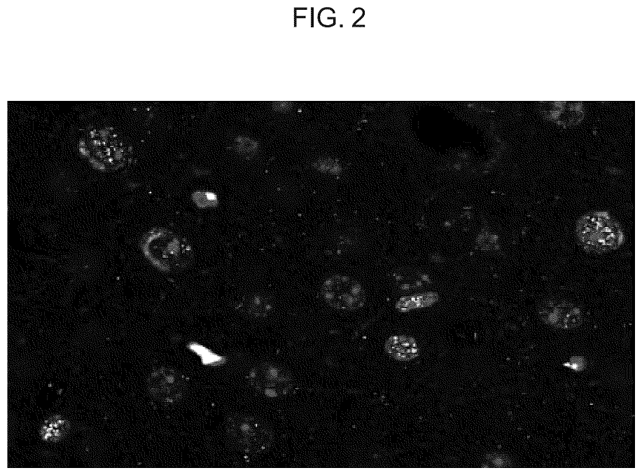

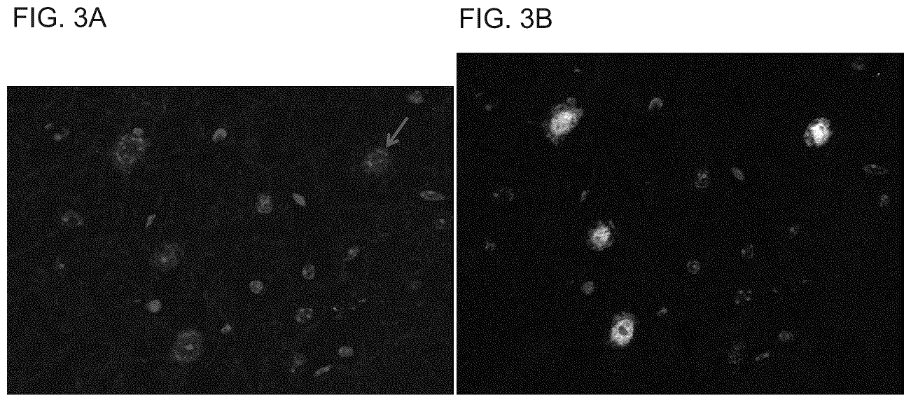

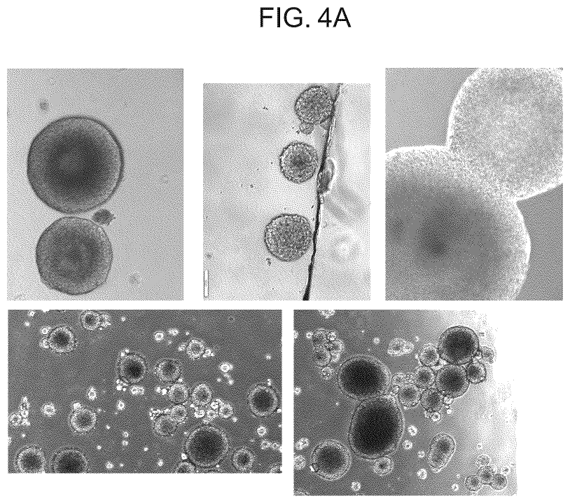

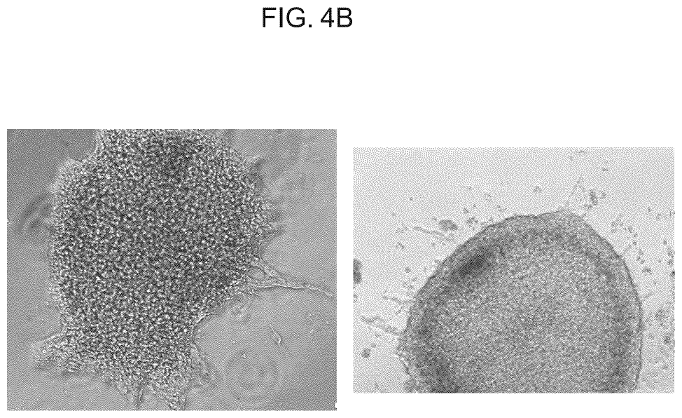

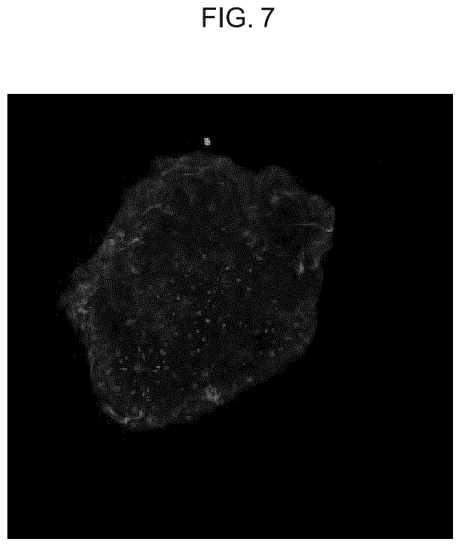

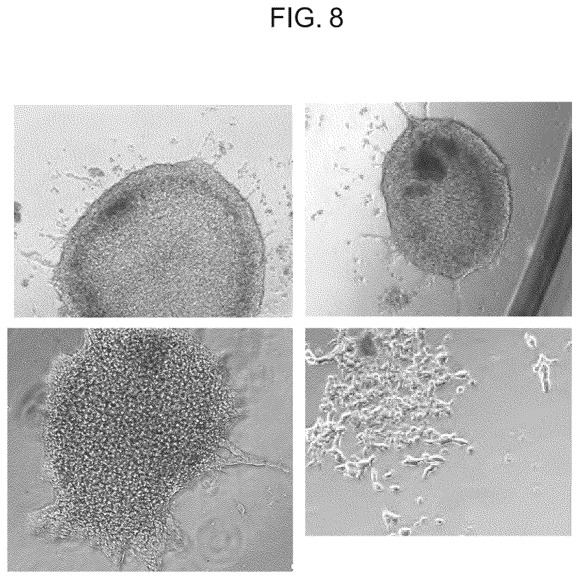

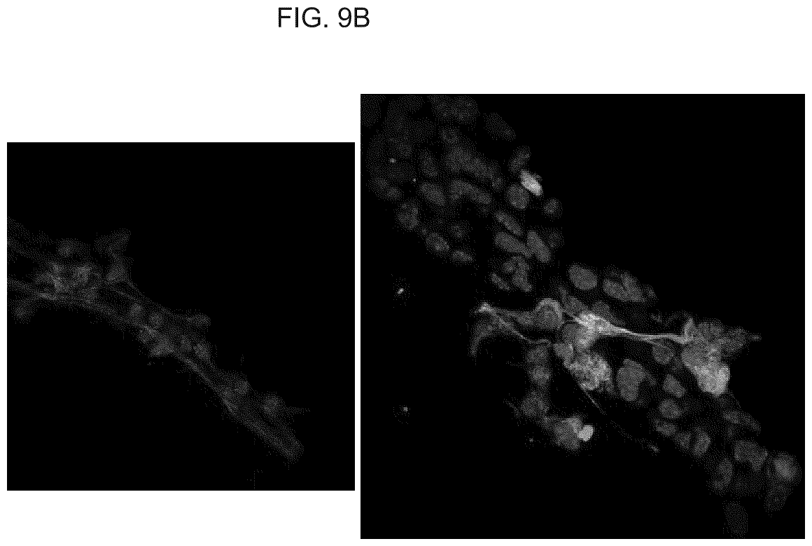

In the examples, this population of cells in the human neural tissue expresses c-kit, also called KIT or CD 117, which is a cytokine receptor that binds cytokine stem cell factor (SCF). SCF signals to cells to divide and grow. In general, c-kit is expressed on the surface of stem cells as well as the progenitor and precursor cell types which are progeny from the stem cells by mitotic division. Therefore, c-kit is a stem cell marker. By immunostaining for c-kit in human neural tissues, the inventors found such c-kit positive cells (FIG. 1A-1B, FIG. 2, FIG. 3A-3B, FIG. 6A-6B, FIG. 7, FIG. 9A-9B). Prior to this discovery, there has been no reported evidence of c-kit positive stem cells in neural tissue. These c-kit positive cells comprise lineage-negative cells, progenitor cells and/or lineage-positive cells. In some embodiments, the lineage-positive cells express beta III tubulin, NeuN and/or GFAP.

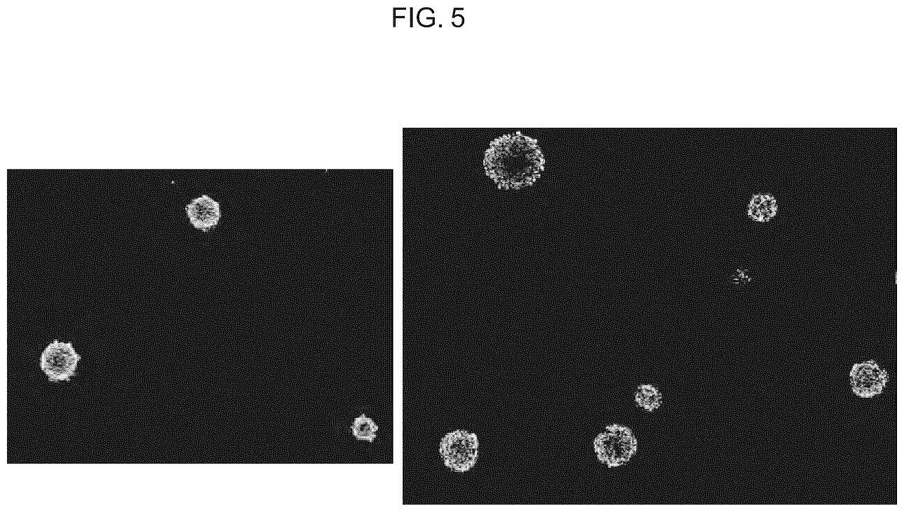

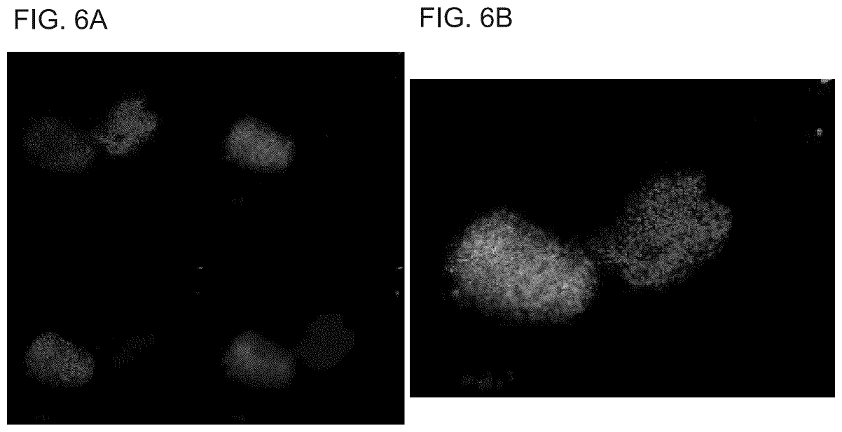

The inventors showed that these c-kit positive NSCs have clonogenic properties. When these cells were isolated and plated in an ultra-low attachment plate and passaged, neurospheres were formed at each passage (FIG. 4A-4B, FIG. 5), thus demonstrating the clonogenic properties of these c-kit positive neural stem cells.

Moreover, c-kit expression alone or in combination with lineage markers was found within the neurospheres (FIG. 6A-6B, FIG. 7, FIG. 9A-9B).

In one embodiment of all aspects of the compositions and methods described, the population of isolated c-kit positive NSCs contains cells that have long-term and short-term regeneration capacities, and committed multipotent, oligopotent, and unipotent progenitors.

Accordingly, as used herein, the term “NSC” refers to a cell with multi-lineage neural differentiation potential and sustained self-renewal activity. “Self renewal” refers to the ability of a cell to divide and generate at least one daughter cell with the identical (e.g., self-renewing) characteristics of the parent cell. The second daughter cell may commit to a particular differentiation pathway. For example, a self-renewing NSC divides and forms one daughter stem cell and another daughter cell committed to differentiation into neuronal and/or glial cells of the neural tissue. A committed progenitor cell has typically lost the self-renewal capacity, and upon cell division produces two daughter cells that display a more differentiated (i.e., restricted) phenotype,

“NCSs,” as used in the methods described herein, therefore, encompasses all pluripotent cells capable of differentiating into several cell types of neural tissue, including, but not limited to, cholinergic neurons, GABAergic (gamma aminobutyric acid) neurons, glutamatergic neurons, dopaminergic neurons, serotonergic neurons, motor neurons, interneurons, astrocytes, oligodendrocytes and/or microglia.

“Neural progenitor cells,” as the term is used herein, refer to the subset of NSC that are committed to a particular neural cell lineage and generally do not self-renew, and can be identified, for example by cell surface markers or intracellular proteins. For example, beta III tubulin or NeuN which indicates commitment to the neuronal cell lineage; or GFAP which indicates commitment to the glial cell lineage. NeuN (neuronal nuclei) is a neuron-specific nuclear protein which is identified by immunoreactivity with a monoclonal antibody, anti-NeuN. NeuN has been identified as Fox-3, a hexaribonucleotide-binding protein 3 that functions as a splicing regulator. Beta III tubulin (also known as class III beta-tubulin or beta-tubulin III) is a microtubule element of the tubulin family found almost exclusively in neurons. In some embodiments of all aspects of the compositions and methods described, NSCs are selected for using one or more of these additional cell surface markers.

The presence of NSC can be determined by any method known in the art, or phenotypically through the detection of cell surface markers using assays known to those of skill in the art or those described in the examples.

### Isolation of NSCs

In some embodiments of all aspects of the compositions and methods described, the NSCs are derived or isolated from neural tissue samples of the following sources: freshly deceased subjects, tissue biopsy from a live subject, or a neural stem cell line. In some embodiments of all aspects of the compositions and methods described, the NSCs are derived ex vivo from other cells, such as induced pluripotent stem cells (IPS cells) or adult pluripotent cells,

In one embodiment of all aspects of the compositions and methods described, the NSC can be isolated using any method known to one of skill in the art or according to the method described herein, for example, fine needle aspiration for a small neural tissue sample from a live subject.

NSC can be isolated from neural tissue samples by any method known in the art. Methods of dissociating individual cells from a tissue sample are known in the art, e.g., in U.S. Pat. No. 7,547,674 and U.S. Patent Application U.S. 2006/0239983, 2009/0148421, and 2009/0180998. These references are herein incorporated by reference in their entirety.

In one embodiment of all aspects of the compositions and methods described, the population of isolated N SCs is isolated by the following method. One skilled in the art would be able to make minor adjustments to the method as needed for neural tissues from different sources. A small piece of neural tissue, a minimum size of at least 1 cubic cm, is enzymatically digested with collagenase to obtain single cells. Small intact cells are resuspended and aggregates of cells are removed with a cell strainer. This cell strainer step is optional. Then the cells are incubated with a mouse c-kit antibody, c-kit positive cells are isolated and collected with immunomagnetic beads coated with anti-mouse IgG.

In one embodiment of all aspects of the compositions and methods described, the isolated c-kit positive cells obtained are then cultured by the following method. One skilled in the art would be able to make minor adjustments to the method as needed. The culture method is used to grow and expand the number of c-kit positive NSCs, The isolated c-kit positive cells are plated in modified F12K medium containing F12 medium (GIBCO, Grand Island, NY) supplemented with 5-10% FBS (GIBCO) and insulin-selenium-transferrin mixture (SIGMA, St. Louis, MO) under standard tissue culture conditions. After reaching confluence, the cells are passaged to several other plates to expand the culture using standard tissue culture protocol of handling the cells.

In some embodiments of all aspects of the compositions and methods described, the NSC from the neural tissues described herein is expanded ex vivo using any method acceptable to those skilled in the art prior to use in the methods described herein. In some embodiments of all aspects of the compositions and methods described, the expanded c-kit positive NSCs are further sorted, fractionated, treated to remove any undesired cells, or otherwise manipulated to treat the patient using any procedure acceptable to those skilled in the art of preparing cells for transplantation. Example of an undesired cell is a malignant cell.

There is typically a very small number of NSCs in a sample of neural tissue, for example, there can be only one or two c-kit positive cell per one million cells. Therefore, expansion of the selected c-kit positive NSCs is often necessary to increase the number of cells required for the therapeutic uses described herein. The greater number of NSCs transplanted in the therapeutic uses described herein increases the success rate of the therapy used therein. The NSCs are used to repair, reconstitute, generate and/or protect from further damage, some of the damaged tissues and cells in the subject's neural tissue. Therefore, more NSCs transplanted means more cells available to repair, reconstitute and generate new neural cells and neural tissue or protect existing neural cells or tissue from further damage. In some embodiments, a success of the transplant therapy can be measured by any method known in the art and those described herein, such as an improvement in the subject's cognitive function, motor function and general health conditions which are known to a physician skilled in the art.

In some embodiments of all aspects of the compositions and methods described, a neural tissue sample comprising NSCs is isolated from a subject and is then further processed, for example, by cell sorting (e.g., FACS), to obtain a population of substantially enriched c-kit positive NSCs. In other embodiments of all aspects of the compositions and methods described, a population of substantially enriched c-kit positive NSCs refers to an in vitro or ex vivo culture of expanded NSCs.

In some embodiments of all aspects of the compositions and methods described, the neural tissue samples from the various sources are frozen samples, such as frozen or cryopreserved prior to extraction or selection of the c-kit positive NSCs. The neural tissue sample is obtained from a subject or other sources described herein and then cryopreserved with cryoprotectant. In another embodiment of all aspects of the compositions and methods described, the population of isolated c-kit NSCs from the neural tissue sample is cryopreserved with cryoprotectant prior to use. In yet another embodiment of all aspects of the compositions and methods described, the population of isolated c-kit NSCs that has been expanded in vitro culture is cryopreserved with cryoprotectant prior to use. Methods of cryopreservation of tissues and cells with cryoprotectant are well known in the art. Further methods for thawing the cryopreserved tissue or cells for use are also well known in the art.

The terms “isolate” and “methods of obtaining or preparing,” as used herein, refer to a process whereby a cell or a population of cells, such as a population of NSCs, is removed from a subject or from a neural tissue sample in which it was originally found. The term “isolated population,” as used herein, refers to a population of cells that has been removed and separated from a biological sample, or a mixed or heterogeneous population of cells found in such a sample. Such a mixed population includes, for example, a population of NSCs obtained from a neural tissue sample. In some embodiments, an isolated population is a substantially pure population of cells as compared to the heterogeneous population from which the cells were isolated or enriched from. In some embodiments, the isolated population is a population of isolated c-kit positive NSCs. In other embodiments of this aspect and all aspects described herein, the isolated population comprises a substantially enriched population of c-kit positive NSCs. In some embodiments, an isolated cell or cell population, such as a population of c-kit positive NSCs, is further cultured in vitro or ex vivo, e.g., in the presence of growth factors or cytokines, to further expand the number of cells in the isolated cell population or substantially c-kit enriched cell population. In one embodiment, the population of c-kit positive NSCs is further cultured in vitro or ex vivo with SCF, IGF-1, HGF, bFGF and/or NGF. Such culture can be performed using any method known to one of skill in the art. In some embodiments, the isolated or substantially enriched c-kit positive NSC populations obtained by the methods disclosed herein are later administered to a second subject, or re-introduced into the subject from which the cell population was originally isolated (e.g., allogeneic transplantation vs. autologous administration).

The term “substantially enriched,” with respect to a particular cell population, refers to a population of cells that is at least about 75%, at least about 80%, at least about 85%, at least about 90%, at least about 95%, at least about 98%, or at least about 99% pure, with respect to the cells making up a total cell population. In other words, the terms “substantially enriched” or “essentially purified”, with regard to a population of c-kit positive NSCs isolated for use in the methods disclosed herein, refers to a population of c-kit positive NSCs that contain fewer than about 25%, fewer than about 20%, fewer than about 15%, fewer than about 10%, fewer than about 9%, fewer than about 8%, fewer than about 7%, fewer than about 6%, fewer than about 5%, fewer than about 4%, fewer than about 3%, fewer than about 2%, fewer than about 1%, or less than 1%, of cells that are not NSC, as defined by the terms herein. Some embodiments of these aspects further encompass methods to expand a population of substantially pure or enriched NSCs, wherein the expanded population of c-kit positive NSCs is also a substantially pure or enriched population of c-kit positive NSCs.

The term “substantially negative,” with respect to a particular marker presence in a cell population, refers to a population of cells that is not more than about 10%, not more than about 8%, not more than about 6%, not more than about 4%, not more than about 2%, not more than about 1% positive for that marker, with respect to the cells making up a total cell population.

The terms “enriching” or “enriched” are used interchangeably herein and mean that the yield (fraction) of cells of one type, such as NSCs for use in the methods described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biological sample, culture, or preparation. A population of c-kit positive NSCs obtained for use in the methods described herein is most preferably at least 60% enriched for c-kit positive NSCs,

In some embodiments, markers specific for NSCs are used to isolate or enrich for these cells. A “marker,” as used herein, describes the characteristics and/or phenotype of a cell. Markers can be used for selection of cells comprising characteristics of interest. Markers will vary with specific cells. Markers are characteristics, whether morphological, functional or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, appearance (e.g., smooth, translucent), and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

Accordingly, as used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markers often provide antigenic determinants to which antibodies can bind to. A cell-surface marker of particular relevance to the methods described herein is CD117 or c-kit. The useful NSCs according to the compositions and method preferably express c-kit or in other words, they are c-kit positive.

A cell can be designated “positive” or “negative” for any cell-surface marker or other intracellular marker, and both such designations are useful for the practice of the methods described herein. A cell is considered “positive” for a cell-surface marker if it expresses the marker on its cell-surface or intracellularly in amounts sufficient to be detected using methods known to those of skill in the art, such as contacting a cell with an antibody that binds specifically to that marker, and subsequently performing flow cytometric analysis of such a contacted cell to determine whether the antibody is bound to the cell. It is to be understood that while a cell can express messenger RNA for a cell-surface marker, in order to be considered positive for the methods described herein, the cell must express the marker on its surface. Similarly, a cell is considered “negative” for a cell-surface marker or other intracellular marker if it does not express the marker in amounts sufficient to be detected using methods known to those of skill in the art, such as contacting a cell with an antibody that binds specifically to that marker and subsequently performing flow cytometric analysis of such a contacted cell to determine whether the antibody is bound to the cell.

In some embodiments of all aspects of the compositions and methods described, the c-kit positive NSCs are negatively selected and the selection uses an agent specific for a cell surface marker. In some embodiments of all aspects of the compositions and methods described, the cell surface marker is a lineage specific marker such as a neuronal cell lineage or a glial cell lineage.

In some embodiments of all aspects of the compositions and methods described, in the context of negative selection, where agents specific for lineage markers are used, all of the agents can comprise the same label or tag, such as a fluorescent tag, and thus all cells positive for that label or tag can be excluded or removed, leaving the lineage marker-negative NSCs, neural progenitor cells and/or neural precursor cells for use in the methods described herein. This is negative selection, selecting for those cells that did not contact with the agents specific for lineage markers.

Accordingly, as defined herein, an “agent specific for a cell-surface marker or other intracellular marker” refers to an agent that can selectively react with or bind to that cell-surface marker or other intracellular marker, but has little or no detectable reactivity to another cell-surface marker, other intracellular marker or antigen. For example, an agent specific for c-kit will not identify or bind to CD49e. Thus, agents specific for cell-surface markers or other intracellular marker recognize unique structural features of the markers. In some embodiments, an agent specific for a marker binds to the marker, but does not cause initiation of downstream signaling events mediated by that marker, for example, a non-activating antibody. Agents specific for cell-surface molecules include, but are not limited to, antibodies or antigen-binding fragments thereof, natural or recombinant ligands, small molecules, nucleic acid sequence and nucleic acid analogues, intrabodies, aptamers, and other proteins or peptides.

In some embodiments of all aspects of the compositions and methods described, the preferred agents specific for cell-surface markers used for isolating NSCs are antibody agents that specifically bind the cell-surface markers, and can include polyclonal and monoclonal antibodies, and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. Accordingly, as used herein, the term “antibody” refers to an intact immunoglobulin or to a monoclonal or polyclonal antigen-binding fragment with the Fc (crystallizable fragment) region or FcRn binding fragment of the Fc region. Antigen-binding fragments may be produced by recombinant DNA techniques or by enzymatic or chemical cleavage of intact antibodies. “Antigen-binding fragments” include, inter alia. Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′)2 and Fv are employed with standard immunological meanings known to those skilled in the art, e.g., in Klein, “Immunology” (John Wiley, New York, N.Y., 1982), Clark, W. R. (1986), in “The Experimental Foundations of Modern Immunology” (Wiley & Sons, Inc., New York); and Roitt, I. (1991) “Essential Immunology”, 7th Ed, (Blackwell Scientific Publications, Oxford). Such antibodies or antigen-binding fragments are available commercially from vendors such as R&D Systems, BD Biosciences, e-Biosciences and Miltenyi, or can be raised against these cell-surface markers or other intracellular marker by methods known to those skilled in the art.

In some embodiments of all aspects of the compositions and methods described, an agent specific for a cell-surface molecule or other intracellular marker, such as an antibody or antigen-binding fragment, is labeled with a tag to facilitate the isolation of the neural stem cells. The terms “label” or “tag”, as used herein, refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods to isolate and enrich for NSCs, neural progenitor cell and neural precursor cells.

The terms “labeled antibody” or “tagged antibody”, as used herein, includes antibodies that are labeled by detectable means and include, but are not limited to, antibodies that are fluorescently, enzymatically, radioactively, and chemiluminescently labeled. Antibodies can also be labeled with a detectable tag, such as c-Myc, HA, VSV-G, HSV, FLAG, V5, or HIS, which can be detected using an antibody specific to the tag, for example, an anti-c-Myc antibody. Various methods of labeling polypeptides and glycoproteins are known in the art and may be used. Non-limiting examples of fluorescent labels or tags for labeling the antibodies for use in the methods of invention include hydroxycoumarin, succinimidyl ester, aminocoumarin, succinimidyl ester, methoxycoumarin, Cascade Blue, Hydrazide, Pacific Blue, maleimide, Pacific Orange, lucifer yellow, NBD, NBD-X, R-phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, ALEXA FLUOR® 350, ALEXA FLUOR® 405, ALEXA FLUOR® 430, ALEXA FLUOR® 488, ALEXA FLUOR® 500, ALEXA FLUOR® 514, ALEXA FLUOR® 532, ALEXA FLUOR® 546, ALEXA FLUOR®555, ALEXA FLUOR® 568, ALEXA FLUOR®594, ALEXA FLUOR® 610, ALEXA FLUOR® 633, ALEXA FLUOR® 647, ALEXA FLUOR® 660, ALEXA FLUOR® 680, ALEXA FLUOR® 700, ALEXA FLUOR® 750, ALEXA FLUOR® 790, Cy2, Cy3, Cy3B, Cy3.5, Cy5, Cy5.5 or Cy7.

In some embodiments of all aspects of the compositions and methods described, a variety of methods to isolate a substantially pure or enriched population of c-kit positive NSCs are available to a skilled artisan, including immunoselection techniques, such as high-throughput cell sorting using flow cytometric methods, affinity methods with antibodies labeled to magnetic beads, biodegradable beads, non-biodegradable beads, and antibodies panned to surfaces including dishes and combination of such methods.

In some embodiments of all aspects of the compositions and methods described, the isolation and enrichment for populations of NSCs can be performed using bead based sorting mechanisms, such as magnetic beads. In such methods, a digested neural tissue sample is contacted with magnetic beads coated with antibodies against one or more specific cell-surface antigens, such as c-kit. This causes the cells in the sample that express the respective antigen to attach to the magnetic beads. After a period of time to allow the c-kit positive cells to bind the beads, the mixture of cell and beads are exposed to a strong magnetic field, such as a column or rack having a magnet. The cells attached to the beads (expressing the cell-surface marker) stay on the column or sample tube, while other cells (not expressing the cell-surface marker) flow through or remain in solution. Using this method, cells can be separated positively or negatively, or using a combination therein, with respect to the particular cell-surface markers.

In some embodiments of all aspects of the compositions and methods described, magnetic activated cell sorting (MACS) strategies are used for isolation and pre-selection of NSCs. In some embodiments, NSCs are isolated in the presence of human plasma or human serum albumin (HSA), such as 2% HSA.

In some preferred embodiments of all aspects of the compositions and methods described, NSCs are isolated or enriched using positive selection for the cell-surface marker c-kit.

As defined herein, “positive selection” refers to techniques that result in the isolation or enrichment of cells expressing specific cell-surface markers or intracellular proteins, while “negative selection” refers to techniques that result in the isolation or enrichment of cells that do not express specific cell-surface markers or intracellular proteins. Negative selection can be performed by any method known in the art. For example, typical negative selection is carried out by removing the cells that do express the marker of interest.

In some embodiments of all aspects of the compositions and methods described, beads can be coated with antibodies by a skilled artisan using standard techniques known in the art, such as commercial bead conjugation kits. In some embodiments, a negative selection step is performed to remove cells expressing one or more lineage markers, followed by fluorescence activated cell sorting to positively select NSCs expressing one or more specific cell-surface markers.

A number of different cell-surface markers have specific expression on specific differentiated cell lineages, and are not expressed by the c-kit positive NSCs isolated for the methods described herein. Accordingly, when agents specific for these lineage cell-markers are contacted with c-kit positive NSCs, the cells will be “negative.”

In some embodiments of all aspects of the compositions and methods described, flow cytometric methods, alone or in combination with magnetic bead based methods, are used to isolate or enrich for c-kit positive NSCs. As defined herein, “flow cytometry” refers to a technique for counting and examining microscopic particles, such as cells and DNA, by suspending them in a stream of fluid and passing them through an electronic detection apparatus. Flow cytometry allows simultaneous multiparametric analysis of the physical and/or chemical parameters of up to thousands of particles per second, such as fluorescent parameters. Modern flow cytometric instruments usually have multiple lasers and fluorescence detectors. Increasing the number of lasers and detectors allows for labeling by multiple antibodies, and can more precisely identify a target population by their phenotypic markers. Certain flow cytometric instruments can take digital images of individual cells, allowing for the analysis of fluorescent signal location within or on the surface of cells.

A common variation of flow cytometric techniques is to physically sort particles based on their properties, so as to purify populations of interest, using “fluorescence-activated cell sorting” As defined herein, “fluorescence-activated cell sorting” or “flow cytometric based sorting” methods refer to flow cytometric methods for sorting a heterogeneous mixture of cells from a single biological sample into one or more containers, one cell at a time, based upon the specific light scattering and fluorescent characteristics of each cell and provides fast, objective and quantitative recording of fluorescent signals from individual cells as well as physical separation of cells of particular interest. Accordingly, in those embodiments when the agents specific for cell-surface markers are antibodies labeled with tags that can be detected by a flow cytometer, fluorescence-activated cell sorting (FACS) can be used in and with the methods described herein to isolate and enrich for populations of NSCs.

### Expansion of NSCs

In some embodiments of all aspects of the compositions and methods described, the population of isolated and substantially enriched c-kit positive NSCs are further expanded to increase in numbers prior to their use in the therapeutic methods described herein.

In some embodiments of all aspects of the compositions and methods described, c-kit positive NSCs isolated or enriched by using the methods and techniques described herein are expanded in culture, i.e., the cell numbers are increased outside the body of the subject, using methods known to one of skill in the art, prior to administration to a subject in need.

In one embodiment of all aspects of the compositions and methods described, the isolated c-kit positive NSCs obtained are expanded in culture according to the following method. One skilled in the art would be able to make minor adjustment to the method as needed. The isolated c-kit positive cells are plated in modified F12K medium containing F12 medium (GIBCO, Grand Island, NY) supplemented with 5-10% FBS (GIBCO) and insulin-selenium-transferrin mixture (SIGMA, St. Louis, MO) under standard tissue culture conditions, e.g., 95% air, 5% CO2, 37° C. After reaching confluence, the cells from one confluent plate are passaged to several other plates to expand the culture using standard tissue culture protocol of handling the cells.

In some embodiments of all aspects of the compositions and methods described, such expansion methods can comprise, for example, culturing the c-kit positive NSCs in serum-free medium supplemented with cytokines and/or growth factors under conditions that cause expansion of NSCs, such as SCF, IGF-1, HGF, bFGF and/or NGF. HGF positively influences cell migration through the expression and activation of matrix metalloproteinase-2. This enzyme family destroys barriers in the extracellular matrix thereby facilitating stem cell movement, homing and tissue restoration. Similarly, insulin-like growth factor-1 (IGF-1) is mitogenic, anti-apoptotic and is necessary for neural stem cell multiplication and differentiation. In a comparable manner, IGF-1 impacts stem cells by increasing their number and protecting their viability. bFGF stimulates the proliferation of all cells of mesodermal origin, and many cells of neuroectodermal, ectodermal and endodermal origin. bFGF is a chemotactic and mitogenic agent for endothelial cells in vitro and induces neural differentiation, survival and regeneration. It has been shown to be crucial in modulating embryonic development and differentiation and it may play a role in the modulation of angiogenesis, tissue repair, embryonic development and neuronal function in vivo. NGF is a neuropeptide primarily involved in the regulation of growth, maintenance, proliferation, and survival of certain target neurons. Numerous biological processes involving NGF have been identified, two of them being the survival of pancreatic beta cells and the regulation of the immune system. In some embodiments of all aspects of the compositions and methods described, the c-kit positive NSCs can further be cultured with factors and/or under conditions aimed at inducing differentiation of the NSCs to neuronal and/or glial cells, such as using serum-free medium supplemented with dexamethasone and/or a combination of growth factors and cytokines.

In other embodiments of all aspects of the compositions and methods described, c-kit positive NSCs are expanded by adapting not more than about 0.5%, nanotechnological or nanoengineering methods, as reviewed in Lu J et al., “A Novel Technology for Hematopoietic Stem Cell Expansion using Combination of Nanofiber and Growth Factors.” Recent Pat Nanotechnol. 2010 4 (2): 125-35. For example, in some embodiments, nanoengineering of stem cell microenvironments can be performed. As used herein, secreted factors, stem cell-neighboring cell interactions, extracellular matrix (ECM) and mechanical properties collectively make up the “stem cell microenvironment”. Stem cell microenvironment nanoengineering can comprise the use of micro/nanopatterned surfaces, nanoparticles to control release growth factors and biochemicals, nanofibers to mimic extracellular matrix (ECM), nanoliter-scale synthesis of arrayed biomaterials, self-assembly peptide system to mimic signal clusters of stem cells, nanowires, laser fabricated nanogrooves, and nanophase thin films to expand NSCs,

In other embodiments of all aspects of the compositions and methods described, the c-kit positive NSCs are genetically manipulated, e.g., transfected with an exogenous nucleic acid. Nanoengineering can be used for the transfection and genetic manipulation in NSCs, such as nanoparticles for in vivo gene delivery, nanoneedles for gene delivery to NSCs, self-assembly peptide system for NSC transfection, nanowires for gene delivery to NSCs, and micro/nanofluidic devices for NSC electroporation.

In other embodiments of all aspects of the compositions and methods described, the c-kit positive NSCs isolated or enriched for use in the methods can be expanded using bioreactors.

The terms “increased,” “increase” or “expand”, when used in the context of NSC expansion, generally mean an increase in the number of NSCs by a statistically significant amount; for the avoidance of any doubt, the terms “increased,” “increase,” “expand” or “expanded,” mean an increase, as compared to a reference level, of at least about 10%, of at least about 15%, of at least about 20%, of at least about 25%, of at least about 30%, of at least about 35%, of at least about 40%, of at least about 45%, of at least about 50%, of at least about 55%, of at least about 60%, of at least about 65%, of at least about 70%, of at least about 75%, of at least about 80%, of at least about 85%, of at least about 90%, of at least about 95%, or up to and including a 100%, or at least about a 2-fold, or at least about a 3-fold, or at least about a 4-fold, or at least about a 5-fold, at least about a 6-fold, or at least about a 7-fold, or at least about a 8-fold, at least about a 9-fold, or at least about a 10-fold increase, or any increase of 10-fold or greater, as compared to a control or reference level. A control/reference sample or level is used herein to describe a population of cells obtained from the same biological source that has, for example, not been expanded using the methods described herein, e.g., at the start of the expansion culture or the initial number of cells added to the expansion culture.

Storage of neural tissue samples and/or neural stem cells

In some embodiments of all aspects of the compositions and methods described, the neural tissue samples are stored prior to use, i.e., prior to the extraction, isolation or selection of the c-kit positive NSCs therein. In some embodiments of all aspects of the compositions and methods described, the digested neural tissue sample is stored prior to extraction or selection of the c-kit positive NSCs therein. In some embodiments of all aspects of the compositions and methods described, the isolated c-kit positive NSCs are stored. In other embodiments of all aspects of the compositions and methods described, the c-kit positive NSCs are first isolated and/or expanded prior to storage. In one embodiment, the storage is by cryopreservation. The NSCs are thawed when needed for the therapeutic methods described herein.

In some embodiments of all aspects of the compositions and methods described, the neural tissue samples or isolated c-kit positive NSCs (expanded or otherwise) are frozen prior to their use in the methods described herein. Freezing the samples can be performed in the presence of one or more different cryoprotectants for minimizing cell damage during the freeze-thaw process. For example, dimethyl sulfoxide (DMSO), trehalose, or sucrose can be used.

### Administration and Uses of NSCs in Regenerative Medicine

Certain embodiments described herein are based on the discovery of somatic stem cells in mouse neural tissue and that these mouse neural stem cells (mNSCs) can repair damaged neural tissues in mice models of neurological diseases or disorders. When mNSCs are placed into a mouse with damaged neural tissue, long-term engraftment of the administered mNSCs can occur and these mNSCs can differentiate into neurons, for example, which can lead to subsequent neuron regeneration and repair. This experiment can indicate whether isolated c-kit positive NSCs can be used for neural tissue regeneration and treatment of neurological diseases or disorders.

Accordingly, provided herein are methods for the treatment and/or prevention of a neurological disease or disorder in a subject in need thereof. As used herein, the term “neurological disease or disorder”, “neurological disease”, “neurological condition” and “neurological disorder” are used interchangeably. Some of these methods involve administering to a subject a therapeutically effective amount of isolated c-kit positive NSCs by injection, by a catheter system, or a combination thereof. In some aspects of these methods, a therapeutically effective amount of isolated c-kit positive NSCs is administered through vessels, directly to the tissue, or a combination thereof. These methods are particularly aimed at therapeutic and prophylactic treatments of human subjects having or at risk for a neurological disease or disorder, e.g., a subject having Alzheimer's disease or multiple sclerosis. The isolated or enriched c-kit positive NSCs described herein can be administered to a selected subject having any neurological disease or disorder or is predisposed to developing a neurological disease or disorder, the administration can be by any appropriate route which results in an effective treatment in the subject. In some embodiments of all aspects of the therapeutic methods described herein, a subject having a neurological disease or disorder is first selected prior to administration of the cells.

The terms “subject”, “patient” and “individual” are used interchangeably herein, and refer to an animal, for example, a human from whom cells for use in the methods described herein can be obtained (i.e., donor subject) and/or to whom treatment, including prophylactic treatment, with the cells as described herein, is provided, i.e., recipient subject. For treatment of those conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or food production mammal, e.g., cow, sheep, pig, and the like.

Accordingly, in some embodiments of the therapeutic methods described herein, a subject is a recipient subject, i.e., a subject to whom the isolated c-kit positive NSCs are being administered, or a donor subject, i.e., a subject from whom a neural tissue sample comprising c-kit positive NSCs are being obtained. A recipient or donor subject can be of any age. In some embodiments, the subject is a “young subject,” defined herein as a subject less than 10 years of age. In other embodiments, the subject is an “infant subject,” defined herein as a subject is less than 2 years of age. In some embodiments, the subject is a “newborn subject,” defined herein as a subject less than 28 days of age. In a preferred embodiment, the subject is a human adult.

In some embodiments of the therapeutic methods described herein, the isolated c-kit positive NSC population being administered comprises allogeneic NSCs obtained from one or more donors. As used herein, “allogeneic” refers to NSCs or neural tissue samples comprising NSCs obtained from one or more different donors of the same species, where the genes at one or more loci are not identical. For example, an isolated c-kit positive NSC population being administered to a subject can be obtained from the neural tissue obtained from one more unrelated donor subjects, or from one or more non-identical siblings or other sources. In some embodiments, syngeneic isolated c-kit positive NSC populations is used, such as those obtained from genetically identical animals, or from identical twins. In other embodiments of this aspect, the isolated c-kit positive NSCs are autologous NSCs. As used herein, “autologous” refers to NSCs or neural tissue samples comprising c-kit positive NSCs obtained or isolated from a subject and being administered to the same subject, i.e., the donor and recipient are the same.

Neurological disease or disorder is any disease or disorder that occurs in the neural tissue or that causes the neural tissue to not work properly. Neurological diseases or disorders can include, but are not limited to, stroke, brain hemorrhage, spinal cord injury, Huntington's disease, Parkinson's disease, Alzheimer's disease, amyotrophic lateral sclerosis (ALS), multiple sclerosis (MS), Batten disease and/or ataxia telangiectasia.

The methods described herein can be used to treat, ameliorate the symptoms, prevent and/or slow the progression of a number of neurological diseases or their symptoms, such as those resulting in pathological damage to neural architecture. The terms “neurological disease or disorder”, “neurological disease”, “neurological condition” and “neurological disorder” are used interchangeably herein and refer to any condition and/or disorder relating to the structure or function of the neural tissue, including the neurons and glial cells. Such neurological diseases include, but are not limited to, stroke, brain hemorrhage, spinal cord injury, Huntington's disease, Parkinson's disease, Alzheimer's disease, amyotrophic lateral sclerosis (ALS), multiple sclerosis (MS), Batten disease and/or ataxia telangiectasia.

In some of these conditions, where inflammation plays a role in the pathology of the condition, therapeutic agents used together with the c-kit NSCs can ameliorate or slow the progression of the condition by reducing damage from inflammation. In other cases, therapeutic agents used together with the c-kit NSCs can act to limit pathogen replication or pathogen-associated neural tissue damage.

As used herein, the terms “administering,” “introducing”, “transplanting” and “implanting” are used interchangeably in the context of the placement of cells, e.g., c-kit positive NSCs, of the invention into a subject, by a method or route which results in at least partial localization of the introduced cells at a desired site, such as a site of injury or repair, such that a desired effect(s) is produced. The cells e.g., c-kit positive NSCs, or their differentiated progeny (e.g., glial cells) can be implanted directly to the neural tissue, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of all aspects of the therapeutic methods described herein, an effective amount of an isolated or enriched population of isolated c-kit positive NSCs is administered directly to the neural tissue of an individual suffering from, for example, stroke by direct injection. In other embodiments of all aspects of the therapeutic methods described herein, the population of isolated and enriched c-kit positive NSCs is administered via an indirect systemic route of administration, such as a catheter-mediated route,

One embodiment of the invention includes use of a catheter-based approach to deliver the injection. The use of a catheter precludes more invasive methods of delivery such as surgically opening the body to access the neural tissue. As one skilled in the art is aware, optimum time of recovery would be allowed by the more minimally invasive procedure, which as outlined here, includes a catheter approach. A catheter approach includes the use of such techniques as the NOGA catheter or similar systems. The NOGA catheter system facilitates guided administration by providing electromechanic mapping of the area of interest, as well as a retractable needle that can be used to deliver targeted injections or to bathe a targeted area with a therapeutic. Any of the embodiments of the present invention can be administered through the use of such a system to deliver injections or provide a therapeutic. One of skill in the art will recognize alternate systems that also provide the ability to provide targeted treatment through the integration of imaging and a catheter delivery system that can be used with the present invention. Information regarding the use of NOGA and similar systems can be found in, for example, Sherman, W. (2003) Basic Appl. Myol, 13 (1): 11-14; Patel, A. N. et al. (2005) The Journal of Thoracic and Cardiovascular Surgery 130 (6): 1631-1638; and Perrin, E. G. et al. (2003) Circulation 107:2294-2302; the text of each of which are incorporated herein in their entirety.

When provided prophylactically, the isolated and enriched c-kit positive NSCs can be administered to a subject in advance of any symptom of a neurological disease or disorder. Accordingly, the prophylactic administration of an isolated or enriched for c-kit positive NSC population serves to prevent a neurological disease or disorder, or further progress of neurological diseases or disorders as disclosed herein.

When provided therapeutically, isolated and enriched c-kit positive NSCs are provided at (or after) the onset of a symptom or indication of a neurological disease or disorder, e.g., upon the onset of Alzheimer's.

As used herein, the terms “treat,” “treatment,” “treating,” or “amelioration” refer to therapeutic treatment, wherein the object is to reverse, alleviate, ameliorate, decrease, inhibit, or slow down the progression or severity of a condition associated with a disease or disorder. The term “treating” includes reducing or alleviating at least one adverse effect or symptom of a condition, disease or disorder associated with a neurological disease, such as, but not limited to, Alzheimer's. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. In some embodiments, “treatment” and “treating” can also mean prolonging survival of a subject as compared to expected survival if the subject did not receive treatment,

As used herein, the term “prevention” refers to prophylactic or preventative measures wherein the object is to prevent or delay the onset of a disease or disorder, or delay the onset of symptoms associated with a disease or disorder. In some embodiments, “prevention” refers to slowing down the progression or severity of a condition or the deterioration of neurological function associated with a neurological disease or disorder.

In another embodiment, “treatment” of a neurological disease or disorder also includes providing relief from the symptoms or side-effects of the disease (including palliative treatment). For example, any improvement in memory, cognitive ability and/or motor function, no matter how slight, would be considered an alleviated symptom. In some embodiments of the aspects described herein, the symptoms or a measured parameter of a disease or disorder are alleviated by at least 5%, at least 10%, at least 20%, at least 30%, at least 40%, at least 50%, at least 60%, at least 70%, at least 80%, or at least 90%, upon administration of a population of isolated and enriched NSCs, as compared to a control or non-treated subject.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions as disclosed herein will be decided by the attending physician within the scope of sound medical judgment. The exact amount required will vary depending on factors such as the type of neurological disease or disorder being treated, degree of damage, whether the goal is treatment or prevention or both, age of the subject, the amount of cells available etc. Thus, one of skill in the art realizes that a treatment may improve the disease condition, but may not be a complete cure for the disease.

In one embodiment of all aspects of the therapeutic methods described, the term “effective amount” as used herein refers to the amount of a population of isolated or enriched for c-kit positive NSCs needed to alleviate at least one or more symptoms of the neurological disease or disorder, and relates to a sufficient amount of pharmacological composition to provide the desired effect, e.g., treat a subject having Parkinson's disease. The term “therapeutically effective amount” therefore refers to an amount of isolated and enriched for c-kit positive NSCs using the therapeutic methods as disclosed herein that is sufficient to effect a particular effect when administered to a typical subject, such as one who has or is at risk for Parkinson's.

In another embodiment of all aspects of the methods described, an effective amount as used herein would also include an amount sufficient to prevent or delay the development of a symptom of the disease, alter the course of a disease symptom (for example, but not limited to, slow the progression of a symptom of the disease), or even reverse a symptom of the disease. The effective amount of c-kit positive cells needed for a particular effect will vary with each individual and will also vary with the type of neurological disease or disorder being addressed. Thus, it is not possible to specify the exact “effective amount”. However, for any given case, an appropriate “effective amount” can be determined by one of ordinary skill in the art using routine experimentation.

In some embodiments of all aspects of the therapeutic methods described, the subject is first diagnosed as having a disease or disorder affecting the neural tissue prior to administering the cells according to the methods described herein. In some embodiments of all aspects of the therapeutic methods described, the subject is first diagnosed as being at risk of developing a neurological disease or disorder prior to administering the cells, e.g., an individual with a genetic disposition for Alzheimer's or who has close relatives with Alzheimer's.

For use in all aspects of the therapeutic methods described herein, an effective amount of isolated c-kit positive NSCs comprises at least 102, at least 5×102, at least 103, at least 5×103, at least 104, at least 5×104, at least 103, at least 2×105, at least 3×105, at least 4×105, at least 5×105, at least 6×105, at least 7×105, at least 8×105, at least 9×105, or at least 1×106 c-kit positive NSCs or multiples thereof per administration. In some embodiments, more than one administration of isolated c-kit positive NSCs is performed to a subject. The multiple administration of isolated c-kit positive NSCs can take place over a period of time. The c-kit positive NSCs can be isolated or enriched for from one or more donors, or can be obtained from an autologous source.

Exemplary modes of administration of NSCs and other agents for use in the methods described herein include, but are not limited to, injection, infusion, inhalation (including intranasal), ingestion, and rectal administration, “Injection” includes, without limitation, intravenous, intraarterial, intraductal, direct injection into the tissue, intraventricular, intracardiac, transtracheal injection and infusion. The phrases “parenteral administration” and “administered parenterally” as used herein, refer to modes of administration other than enteral and topical administration, usually by injection, and includes, without limitation, intravenous, intraventricular, intracardiac, transtracheal injection and infusion. In some embodiments, c-kit positive NSCs can be administered by catheter into the tissue.

In preferred embodiments of all aspects of the therapeutic methods described, an effective amount of isolated c-kit positive NSCs is administered to a subject by injection. In other embodiments, an effective amount of isolated c-kit positive NSCs is administered to a subject by a catheter-mediated system. In other embodiments, an effective amount of isolated c-kit positive NSCs is administered to a subject through vessels, directly to the tissue, or a combination thereof.

In some embodiments of all aspects of the therapeutic methods described, an effective amount of isolated and enriched c-kit positive NSCs is administered to a subject by systemic administration, such as intravenous administration.

The phrases “systemic administration,” “administered systemically”, “peripheral administration” and “administered peripherally” as used herein refer to the administration of population of NSCs other than directly into the neural tissue, such that it enters, instead, the subject's circulatory system.

In some embodiments of all aspects of the therapeutic methods described, one or more routes of administration are used in a subject to achieve distinct effects. For example, isolated or enriched population of c-kit positive NSCs are administered to a subject by both direct injection and catheter-mediated routes for treating or repairing damaged neural tissue. In such embodiments, different effective amounts of the isolated or enriched c-kit positive NSCs can be used for each administration route.

In some embodiments of all aspects of the therapeutic methods described, the methods further comprise administration of one or more therapeutic agents, such as a drug or a molecule, that can enhance or potentiate the effects mediated by the administration of the isolated or enriched c-kit positive NSCs, such as enhancing homing or engraftment of the NSCs, increasing repair of neural cells, or increasing growth and regeneration of neural cells. The therapeutic agent can be a protein (such as an antibody or antigen-binding fragment), a peptide, a polynucleotide, an aptamer, a virus, a small molecule, a chemical compound, a cell, a drug, etc.

As defined herein, “vascular regeneration” refers to de novo formation of new blood vessels or the replacement of damaged blood vessels (e.g., capillaries) after injuries or traumas, as described herein, including but not limited to, neurological disease, “Angiogenesis” is a term that can be used interchangeably to describe such phenomena.

In some embodiments of all aspects of the therapeutic methods described, the methods further comprise administration of c-kit positive NSCs together with growth, differentiation, and angiogenesis agents or factors that are known in the art to stimulate cell growth, differentiation, and angiogenesis in the neural tissue. In some embodiments, any one of these factors can be delivered prior to or after administering the compositions described herein. Multiple subsequent delivery of any one of these factors can also occur to induce and/or enhance the engraftment, differentiation and/or angiogenesis. Suitable growth factors include but are not limited to transforming growth factor-beta (TGFβ), vascular endothelial growth factor (VEGF), platelet derived growth factor (PDGF), angiopoietins, epidermal growth factor (EGF), bone morphogenic protein (BMP), basic fibroblast growth factor (bFGF), nerve growth factor (NGF), insulin and 3-isobutyl-1-methylxasthine (IBMX). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, PA: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag, and these are hereby incorporated by reference in their entirety.

In one embodiment, the composition can include one or more bioactive agents to induce healing or regeneration of damaged tissue, such as recruiting blood vessel forming cells from the surrounding tissues to provide connection points for the nascent vessels. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. Other bioactive agents can promote increased mitosis for cell growth and cell differentiation.

A great number of growth factors and differentiation factors are known in the art to stimulate cell growth and differentiation of stem cells and progenitor cells. Suitable growth factors and cytokines include any cytokines or growth factors capable of stimulating, maintaining, and/or mobilizing progenitor cells. They include but are not limited to stem cell factor (SCF), granulocyte-colony stimulating factor (G-CSF), granulocyte-macrophage stimulating factor (GM-CSF), stromal cell-derived factor-1, steel factor, vascular endothelial growth factor (VEGF), TGFβ, platelet derived growth factor (PDGF), angiopoietins (Ang), epidermal growth factor (EGF), bone morphogenic protein (BMP), fibroblast growth factor (FGF), hepatocyte growth factor (HGF), insulin-like growth factor (IGF-1), nerve growth factor (NGF), interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor a. Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, PA: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In one embodiment of all aspects of the therapeutic methods described, the composition described is a suspension of NSCs in a suitable physiologic carrier solution such as saline. The suspension can contain additional bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof.

In certain embodiments of all aspects of the therapeutic methods described, the bioactive agent is a “pro-angiogenic factor,” which refers to factors that directly or indirectly promote new blood vessel formation. The pro-angiogenic factors include, but are not limited to epidermal growth factor (EGF), E-cadherin, VEGF, angiogenin, angiopoietin-1, fibroblast growth factors: acidic (aFGF) and basic (bFGF), fibrinogen, fibronectin, heparanase, hepatocyte growth factor (HGF), angiopoietin, hypoxia-inducible factor-1 (HIF-1), insulin-like growth factor-1 (IGF-1), IGF, BP-3, platelet-derived growth factor (PDGF), VEGF-A, VEGF-C, pigment epithelium-derived factor (PEDF), vascular permeability factor (VPF), vitronection, leptin, trefoil peptides (TFFs), CYR61 (CCN1), NOV (CCN3), leptin, midkine, placental growth factor platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), c-Myc, granulocyte colony-stimulating factor (G-CSF), stromal derived factor 1 (SDF-1), scatter factor (SF), osteopontin, stem cell factor (SCF), matrix metalloproteinases (MMPs), thrombospondin-1 (TSP-1), pleitrophin, proliferin, follistatin, placental growth factor (PIGF), midkine, platelet-derived growth factor-BB (PDGF), and fractalkine, and inflammatory cytokines and chemokines that are inducers of angiogenesis and increased vascularity, e.g., interleukin-3 (IL-3), interleukin-8 (1L-8). CCL2 (MCP-1), interleukin-8 (IL-8) and CCL5 (RANTES). Suitable dosage of one or more therapeutic agents can include a concentration of about 0.1 to about 500 ng/ml, about 10 to about 500 ng/ml, about 20 to about 500 ng/ml, about 30 to about 500 ng/ml, about 50 to about 500 ng/ml, or about 80 ng/ml to about 500 ng/ml. In some embodiments, the suitable dosage of one or more therapeutic agents is about 10, about 25, about 45, about 60, about 75, about 100, about 125, about 150, about 175, about 200, about 225, about 250, about 275, about 300, about 325, about 350, about 375, about 400, about 425, about 450, about 475, or about 500 ng/ml. In other embodiments, suitable dosage of one or more therapeutic agents is about 0.6, about 0.7, about 0.8, about 0.9, about 1.0, about 1.5, or about 2.0 μg/ml.

In some embodiments of all aspects of the therapeutic methods described, the methods further comprise administration of one or more surfactants as therapeutic agents, or may be used in combination with one or more surfactant therapies. Surfactant, as used herein, refers to any surface active agent, including but not limited to wetting agents, surface tension depressants, detergents, dispersing agents and emulsifiers. Particularly preferred are those that from a monomolecular layer over pulmonary alveolar surfaces, including but not limited to lipoproteins, lecithins, phosphatidylglycerol (PG), dipalmitoyl-phosphatidyl choline (DPPG), apoprotein A, apoprotein B, apoprotein C, apoprotein D, palmitoyl oleoyl, phosphatidyl glycerol palmitic and sphygomyelins. Exemplary surfactants include, but are not limited to surfactant protein A, surfactant protein B, surfactant protein C, surfactant protein D, and mixtures and combinations thereof. Commercially available surfactants include, but are not limited to, KL-4, SURVANTA®, bovine lipid extract surfactant (BLES), INFASURF® (CALFACTANT®), CUROSURF®, HL-10, AEROSURF®, SUBOXONE®, ALVEOFACT®, SURFAXIN®, VENTICUTE®, PUMACTANT®/ALEC, and EXOSURF®.

In some embodiments of all aspects of the therapeutic methods described, administration of one or more other standard therapeutic agents can be combined with the administration of the enriched c-kit positive NSCs to treat neurological diseases or disorders, e.g., stroke or Parkinson's, including the use of anticholinergic agents, β-2-adrenoreceptor agonists, such as formoterol or salmeterol, corticosteroids, antibiotics, anti-oxidation, antihypertension agents, nitric oxide, caffeine, dexamethasone, and 1L-10 or other cytokines. In some embodiments, the included standard therapeutic agents are used for treating the symptoms of the neurological disease,

For example, the use of c-kit positive NSCs in the methods described herein to treat, ameliorate or slow the progression of a condition such as Parkinson's can be optionally combined with other suitable treatments or therapeutic agents. For Parkinson's, this includes, but is not limited to, Levodopa, Carbidopa-levodopa, monoamine oxidase B inhibitors, Catechol-O-methyltransferase (COMT) inhibitors, anticholinergics, amantadine, surgical procedures and/or exercise, or any combination therein.

In some embodiments of all aspects of the therapeutic methods described, the standard therapeutic agents are those that have been described in detail, see, e.g., Harrison's Principles of Internal Medicine, 15.sup.th edition, 2001, E. Braunwald, et al., editors, McGraw-Hill, New York, N.Y., ISBN 0-07-007272-8, especially chapters 252-265 at pages 1456-1526; Physicians Desk Reference 54.sup.th edition, 2000, pages 303-3251, ISBN 1-56363-330-2, Medical Economics Co., Inc., Montvale, N.J. Treatment of any neurological disease or disorder can be accomplished using the treatment regimens described herein. For chronic conditions, intermittent dosing can be used to reduce the frequency of treatment. Intermittent dosing protocols are as described herein.

For the clinical use of the methods described herein, isolated or enriched populations of enriched c-kit positive NSCs described herein can be administered along with any pharmaceutically acceptable compound, material, carrier or composition which results in an effective treatment in the subject. Thus, a pharmaceutical formulation for use in the methods described herein can contain an isolated or enriched population of c-kit positive NSCs in combination with one or more pharmaceutically acceptable ingredients.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent, media (e.g., stem cell media), encapsulating material, manufacturing aid (e.g., lubricant, talc magnesium, calcium or zinc stearate, or stearic acid), or solvent encapsulating material, involved in maintaining the activity of, carrying, or transporting the isolated or enriched populations of NSCs from one organ, or portion of the body, to another organ, or portion of the body.

Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose, (2) phosphate buffered solutions; (3) pyrogen-free water; (4) isotonic saline; (5) malt; (6) gelatin; (7) lubricating agents, such as magnesium stearate, sodium lauryl sulfate and talc; (8) excipients, such as cocoa butter and suppository waxes, (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar, (14) buffering agents, such as magnesium hydroxide and aluminum hydroxide; (15) alginic acid; (16) cellulose, and its derivatives, such as sodium carboxymethyl cellulose, methyl cellulose, ethyl cellulose, microcrystalline cellulose and cellulose acetate; (17) powdered tragacanth; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL, (24) C2-C12 alcohols, such as ethanol; (25) starches, such as corn starch and potato starch; and (26) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Definitions

Unless otherwise defined, all technical and scientific terms used herein have the same meaning as commonly understood by one of ordinary skill in the art to which this invention belongs. Certain terms employed herein, in the specification, examples and claims are collected here.

As used herein, in vivo (Latin for “within the living”) refers to those methods using a whole, living organism, such as a human subject. As used herein, “ex vivo” (Latin: out of the living) refers to those methods that are performed outside the body of a subject, and refers to those procedures in which an organ, cells, or tissue are taken from a living subject for a procedure, e.g., isolating c-kit positive NSCs from neural tissue obtained from a donor subject, and then administering the isolated c-kit positive NSCs sample to a recipient subject. As used herein, “in vitro” refers to those methods performed outside of a subject, such as an in vitro cell culture experiment. For example, isolated c-kit positive NSCs can be cultured in vitro to expand or increase the number of c-kit positive NSCs, or to direct differentiation of the NSCs to a specific lineage or cell type, e.g., glial cells, prior to being used or administered according to the methods described herein.

The term “pluripotent” as used herein refers to a cell with the capacity, under different conditions, to commit to one or more specific cell type lineage and differentiate to more than one differentiated cell type of the committed lineage, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

The term “progenitor” cell are used herein refers to cells that have a cellular phenotype that is more primitive (i.e., is at an earlier step along a developmental pathway or progression than is a fully differentiated or terminally differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. Progenitor cells give rise to precursor cells of specific determinate lineage, for example, certain neural progenitor cells divide to give neuronal cell lineage precursor cells. These precursor cells divide and give rise to many cells that terminally differentiate to, for example, dopaminergic neurons.

The term “precursor” cell is used herein refers to a cell that has a cellular phenotype that is more primitive than a terminally differentiated cell but is less primitive than a stem cell or progenitor cell that is along its same developmental pathway. A “precursor” cell is typically progeny cells of a “progenitor” cell which are some of the daughters of “stem cells”. One of the daughters in a typical asymmetrical cell division assumes the role of the stem cell.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that the cell can be distinguished from other cells. Exemplary distinguishing embryonic stem cell characteristics include, without limitation, gene expression profile, proliferative capacity, differentiation capacity, karyotype, responsiveness to particular culture conditions, and the like,

The term “adult stem cell” is used to refer to any multipotent stem cell derived from non-embryonic tissue, including fetal, juvenile, and adult tissue. In some embodiments, adult stem cells can be of non-fetal origin. Stem cells have been isolated from a wide variety of adult tissues including blood, bone marrow, brain, olfactory, epithelium, skin, neural tissue, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and neural stem cells. As indicated above, stem cells have been found resident in virtually every tissue. Accordingly, the present invention appreciates that stem cell populations can be isolated from virtually any animal tissue.

In the context of cell ontogeny, the adjective “differentiated” or “differentiating” is a relative term meaning a “differentiated cell” is a cell that has progressed further down the developmental pathway than the cell it is being compared with. Thus, stem cells can differentiate to lineage-restricted precursor cells (such as a neural stem cell), which in turn can differentiate into other types of precursor cells further down the pathway (such as a neuronal or glial precursor), and then to an end-stage differentiated cell, which plays a characteristic role in a certain tissue type, and may or may not retain the capacity to proliferate further.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as a neural stem cell) in a cellular differentiation process. Without wishing to be limited to theory, a pluripotent stem cell in the course of normal ontogeny can differentiate first to a neuron or glial cell. Further differentiation of a neural stem cell leads to the formation of the various neural cell types, including cholinergic neurons, GABAergic neurons, glutamatergic neurons, dopaminergic neurons, serotonergic neurons, motor neurons, interneurons, astrocytes, oligodendrocytes and/or microglia.

As used herein, the term “somatic cell” refers to any cell forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

As used herein, the term “adult cell” refers to a cell found throughout the body after embryonic development.

The term “phenotype” refers to one or a number of total biological characteristics that define the cell or organism under a particular set of environmental conditions and factors, regardless of the actual genotype. For example, the expression of cell surface markers in a cell.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The terms “renewal” or “self-renewal” or “proliferation” are used interchangeably herein, are used to refer to the ability of stem cells to renew themselves by dividing into the same non-specialized cell type over long periods, and/or many months to years.

In some instances, “proliferation” refers to the expansion of cells by the repeated division of single cells into two identical daughter cells.

The term “lineages” is used herein describes a cell with a common ancestry or cells with a common developmental fate.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “tissue” refers to a group or layer of specialized cells which together perform certain special functions. The term “tissue-specific” refers to a source of cells from a specific tissue.

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” typically means a decrease by at least about 5%-10% as compared to a reference level, for example a decrease by at least about 20%, or at least about 30%, or at least about 40%, or at least about 50%, or at least about 60%, or at least about 70%, or at least about 80%, or at least about 90% decrease (i.e., absent level as compared to a reference sample), or any decrease between 10-90% as compared to a reference level. In the context of treatment or prevention, the reference level is a symptom level of a subject in the absence of administering a population of c-kit positive NSCs.

The terms “increased”, “increase” or “enhance” are all used herein to generally mean an increase by a statically significant amount; for the avoidance of any doubt, the terms “increased”, “increase” or “enhance” means an increase of at least 10% as compared to a reference level, for example an increase of at least about 20%, or at least about 30%, or at least about 40%, or at least about 50%, or at least about 60%, or at least about 70%, or at least about 80%, or at least about 90% increase or more, or any increase between 10-90% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of c-kit positive NSC expansion in vitro, the reference level is the initial number of c-kit positive NSCs isolated from the neural tissue sample.

The term “express at minimal levels” refers to the limited expression of neural markers such as beta Ill tubulin, NeuN and/or GFAP in isolated c-kit positive neural stem cells as measured by qRT-PCR, FACS, immunoprecipitation, Western blotting, ELISA, microarray, Nanostring, mass spectrometry or other molecular quantitation techniques known in the art. Minimal levels of expression of neuronal and/or glial markers typically mean that each marker is expressing at not more than about 10%, not more than about 8%, not more than about 6%, not more than about 4%, not more than about 2%, not more than about 1% positive for that marker or less relative to c-kit expression, as determined by a molecular assay known to one skilled in the art.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

The term “consisting of refers to compositions, methods, and respective components thereof as described herein, which are exclusive of any element not recited in that description of the embodiment.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13:9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005) and Animal Cell Culture Methods (Methods in Cell Biology, Vol. 57, Jennie P. Mather and David Barnes editors, Academic Press, 1st edition, 1998) which are all herein incorporated by reference in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

This invention is further illustrated by the following examples which should not be construed as limiting. The contents of all references cited throughout this application, as well as the figures are incorporated herein by reference.

Those skilled in the art will recognize, or be able to ascertain using not more than routine experimentation, many equivalents to the specific embodiments of the invention described herein, different culture medium and supplements can be used to culture expand the isolated cells. One skilled in the art would be able to perform tests to evaluate the choice of culture medium and supplements. Such equivalents are intended to be encompassed by the following claims.

The references cited herein and throughout the specification are incorporated herein by reference.

### EXAMPLES

The inventors have employed the stem cell antigen c-kit as a marker for the identification and characterization of neural primitive cells. The c-kit epitope was used to help uncover a pool of neural stem cells (NSCs) which are self-renewing, clonogenic and multipotent. These NSCs are able to regenerate into cells that comprise lineage-negative cells, progenitor cells, and/or lineage-positive cells. The lineage-positive cells express beta III tubulin, NeuN or GFAP.

### Materials and Methods

### Mouse NSCs

Stem cells were obtained from the dentate gyrus (DG) and the sub ventricular zone (SVZ) of mouse brain samples. These regions were chosen because it has been reported that neurogenesis occurs in these anatomical areas.

For the isolation of mouse neural stem cells (mNSCs), tissue fragments were dissociated employing an adapted protocol developed in the inventors' laboratory for the collection and expansion of human cardiac stem cells. Tissue fragments were subjected to mechanical and enzymatic dissociation through repeated pipetting and exposure to a solution containing proteases to obtain a single cell suspension. Cells were sorted with magnetic immunobeads for c-kit (Miltenyi) and after sorting, cell phenotype was defined by immunocytochemistry. Putative mouse NSCs were then cultured in F12 medium (Gibco) supplemented with 5-10% FBS (Gibco) and insulin-selenium-transferrin mixture (Sigma). For immunocytochemistry, when possible, primary antibodies were directly labeled with fluorochromes (Molecular Probes) to avoid cross-reactivity. Immunolabeling was analyzed by confocal microscopy.

### Cloning Assay

Unsorted isolated neural cells were plated at limiting dilution in ultra-low attachment dishes to favor the formation of multicellular aggregates, i.e. neurospheres. Neurospheres were then disaggregated and single cell suspensions were plated again (subcloning) several times in order to obtain clonal neurospheres highly enriched in stem cells. The formation of floating spheres indicates that progenitor cells are present in the preparation.

Differentiation of mNSCs

The phenotype of the cells contained in the subcloned neurospheres was defined by immunocytochemistry. The expression of lineage markers specific of different neural cell types was evaluated.

### Example 1

Identification of c-Kit-Positive Cells in Tissue Sections of Mouse Brain

Tissue samples of mouse brain were immunolabeled to determine whether c-kit-positive cells were present in neural tissue. Cells were analyzed by immunohistochemistry using antibodies against c-kit and against GFAP (a marker for astrocytes), Sox2 (a progenitor cell marker), beta III tubulin (neuron-specific marker) and NeuN (neuron-specific marker).

The expression of c-kit was detected in vivo in cells round in shape and small in size. These cells do not express markers of lineage commitment (FIG. 1). FIG. 1A shows c-kit positive (green) cells in the dentate gyrus that are negative for GFAP (in red), a marker for astrocytes. Similarly, FIG. 1B shows c-kit positive (red) cells that are negative for GFAP (in green). Moreover, neural c-kit positive cells display progenitor cell markers, a finding that supports the view of the primitive state of this cell pool (FIG. 2). FIG. 2 shows c-kit positive (green) cells that are also positive for the progenitor cell marker Sox2 (white dots).

At times, c-kit was found in association with lineage markers of neural cells. These observations suggest that a linear relationship exists between c-kit positive cells and differentiated brain cells (FIG. 3). FIG. 3A-3B shows that c-kit (green) is expressed together with beta III tubulin (red, FIG. 3A) and NeuN (white, FIG. 3B).

### Example 2

### The Clonogenicity and Multipotency of Neural Stem Cells

The clonogenicity of neural stem cells is typically demonstrated by implementing a neurosphere assay. The long-term ability to generate spheres with passaging results in the selection of the true stem cells in the pool, c-kit expression alone or in combination with lineage markers was found within the spheres. These observations provide evidence of the clonogenicity and multipotency of neural c-kit positive cells.

FIGS. 4A and 4B provide examples of neurospheres derived from unsorted cells after 7-14 days in culture.

At passage 2 following stimulation with the ligand of the c-kit receptor, SCF, the cell clusters appear compact and well-separated (FIG. 5). Immunolabeling of neurospheres at passage 2 reveal one neurosphere that expresses c-kit (green), NeuN (gray) and GFAP (red), while the other neurosphere is negative for all three markers (FIG. 6A, individual marker signals and DAPI stain; FIG. 6B, merge of the three marker signals and DAPI stain).

At passage 4, the core of the neurosphere contains c-kit positive (green), lineage-negative cells while the outer layer of the neurosphere expresses the neuronal marker GFAP (red) (FIG. 7).

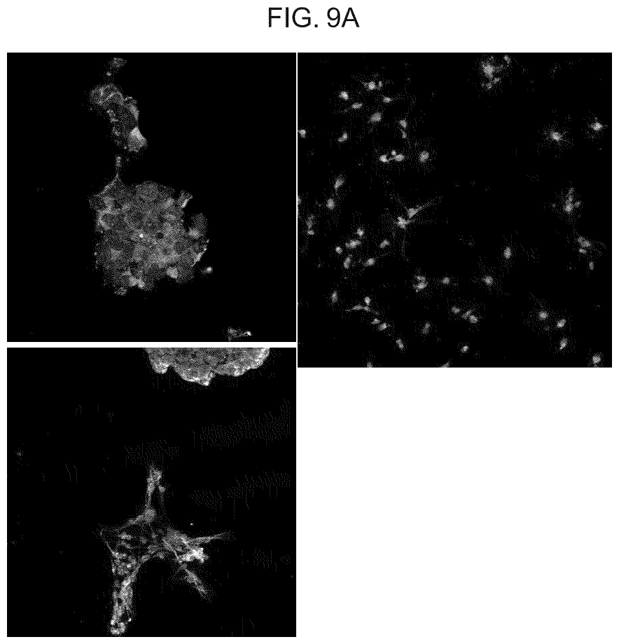

At passage 4 neurospheres were transferred to adherent dishes (FIG. 8) and subsequently stained. The images in FIGS. 9A and 9B are c-kit positive (green) cells partly co-expressing lineage markers of neurons (GFAP, red; NeuN, gray).

What is claimed is:

1. A method of treating or preventing a neurological disease or disorder in a subject in need thereof comprising administering isolated neural stem cells to the subject, wherein the neural stem cells are isolated from a neural tissue specimen and are c-kit positive.

2. The method of claim 1, wherein the neural stem cells are adult neural stem cells.

3. The method of claim 1, wherein the neural stem cells are from the dentate gyrus of the neural tissue specimen.

4. The method of claim 1, wherein the neural stem cells are from the subventricular zone of the neural tissue specimen.

5. The method of claim 1, wherein the neural stem cells comprise lineage-negative cells.

6. The method of claim 1, wherein the neural stem cells comprise progenitor cells.

7. The method of claim 6, wherein the progenitor cells express Sox2.

8. The method of claim 1, wherein the neural stem cells comprise lineage-positive cells.

9. The method of claim 8, wherein the lineage-positive cells express beta III tubulin, NeuN or glial fibrillary acidic protein (GFAP).

10. The method of claim 1, wherein said isolated neural stem cells are expanded in culture prior to administration to the subject.

11. The method of claim 1, wherein the isolated neural stem cells are exposed to one or more cytokines and/or growth factors prior to administration to the subject.

12. The method of claim 1, wherein the isolated neural stem cells are exposed to Stem Cell Factor (SCF), insulin-like growth factor 1 (IGF-1), hepatocyte growth factor (HGF), basic fibroblast growth factor (bFGF) and/or nerve growth factor (NGF) prior to administration to the subject.

13. The method of claim 1, wherein the neural tissue specimen is obtained from the subject.

14. The method of claim 1, wherein the isolated neural stem cells are administered to the subject through vessels or directly to the tissue.

15. The method of claim 1, wherein the isolated neural stem cells are administered to the subject by injection and/or by a catheter system.

16. The method of claim 1, wherein the neurological disease or disorder comprises stroke, brain hemorrhage, spinal cord injury and/or neurodegenerative diseases.

17. The method of claim 16, wherein the neurodegenerative disease comprises Huntington's disease, Parkinson's disease, Alzheimer's disease, amyotrophic lateral sclerosis (ALS), multiple sclerosis (MS), Batten disease and/or ataxia telangiectasia.

18. A method of isolating resident neural stem cells from neural tissue comprising:
(a) culturing a tissue specimen from said neural tissue in culture, thereby forming a tissue explant;
(b) selecting cells from the cultured explant that are c-kit positive, and
(c) isolating said c-kit positive cells, wherein said isolated c-kit positive cells are resident neural stem cells.

19. The method of claim 18, wherein said isolated c-kit positive cells are from the dentate gyrus of the neural tissue.

20. The method of claim 18, wherein said isolated c-kit positive cells are from the sub ventricular zone of the neural tissue.

21. The method of claim 18, wherein said isolated c-kit positive cells comprise lineage-negative cells.

22. The method of claim 18, wherein said isolated c-kit positive cells comprise progenitor cells.

23. The method of claim 18, wherein the progenitor cells express Sox2.

24. The method of claim 18, wherein said isolated c-kit positive cells comprise lineage-positive cells.

25. The method of claim 18, wherein the lineage-positive cells express beta III tubulin, NeuN and/or GFAP.

26. The method of claim 18, further comprising expanding said isolated c-kit positive cells in culture.

27. The method of claim 18, wherein said isolated c-kit positive cells are clonogenic, multipotent and self-renewing.

28. The method of claim 18, further comprising exposing said isolated c-kit positive cells to one or more cytokines and/or growth factors in culture.

29. The method of claim 18, further comprising exposing said isolated c-kit positive cells to Stem Cell Factor (SCF), IGF-1, HGF, bFGF and/or NGF in culture.

30. A method of repairing and/or regenerating damaged neural tissue in a subject in need thereof comprising: extracting neural stem cells from healthy neural tissue; culturing and expanding said neural stem cells, said neural stem cells being c-kit positive stem cells; and administering a dose of said extracted and expanded neural stem cells to an area of damaged neural tissue in the subject effective to repair and/or regenerate the damaged neural tissue.

31. The method of claim 30, wherein the extracted and expanded c-kit positive stem cells are from the dentate gyrus of the healthy neural tissue.

32. The method of claim 30, wherein the extracted and expanded c-kit positive stem cells are from the subventricular zone of the healthy neural tissue.

33. The method of claim 30, wherein the extracted and expanded c-kit positive stem cells comprise lineage-negative cells.

34. The method of claim 30, wherein the extracted and expanded c-kit positive stem cells comprise progenitor cells.

35. The method of claim 30, wherein the extracted and expanded c-kit positive stem cells comprise lineage-positive cells.

36. The method of claim 35, wherein the lineage-positive cells express beta III tubulin, NeuN and/or GFAP.

37. The method of claim 30, wherein the extracted and expanded c-kit positive stem cells are exposed to one or more cytokines and/or growth factors prior to administration to the damaged neural tissue.

38. The method of claim 37, wherein the extracted and expanded c-kit positive stem cells are exposed to Stem Cell Factor (SCF), IGF-1, HGF, bFGF and/or NGF prior to administration to the damaged neural tissue.

39. The method of claim 30, wherein the extracted and expanded c-kit positive stem cells are administered by catheter-mediated or direct injection.

40. The method of claim 30, wherein the neural stem cells are autologous.

41. The method of claim 30, wherein the neural stem cells are allogeneic.
